(19) United States
(12) Patent Application Publication
(10) Pub. No.: US 2010/0293664 A1
(43) Pub. Date: Nov. 18, 2010

(54) CONTROL OF PLANT GENE EXPRESSION

(76) Inventors: Sathish Puthigae, Auckland (NZ); Jonathan Robert Phillips, Chesferfield, MO (US); Nimali Piyushika Withana, Carlton (AU); Claudia Jeannette Smith-Espinoza, Chesterfield, MO (US); Catherine Jane Bryant, Auckland (NZ); Shivendra Bajaj, Auckland (NZ); Kerry Robert Templeton, Auckland (NZ)

Correspondence Address: GREENLEE SULLIVAN P.C., 4875 PEARL EAST CIRCLE, SUITE 200, BOULDER, CO, 80301, US

(21) Appl. No.: 12/594,585

(22) PCT Filed: Apr. 3, 2008
(86) PCT No.: PCT/NZ08/00071

§ 371 Date: May 18, 2010

Related U.S. Application Data
(60) Provisional application No. 60/909,886, filed on Apr. 3, 2007.

(51) Int. Cl. A01H 5/00; C12N 15/82; C07H 21/04; C12N 15/63; C12N 5/10

(52) U.S. Cl. 800/278; 800/298; 536/24.1; 435/320.1; 435/419; 435/468

(57) ABSTRACT

This invention provides an isolated promoter polynucleotide comprising the sequence of SEQ ID NO:1, and variants and fragments thereof capable of controlling transcription of an operably linked polynucleotide in a plant. The invention also provides the constructs, vectors, host cells, plant cells and plants transformed with the isolated promoter polynucleotide. The invention also provides methods of use of the isolated promoter polynucleotide.

### TECHNICAL FIELD

The present invention relates to the isolation and use of the polynucleotides for the control of gene expression in plants.

### BACKGROUND ART

An important for goal for agriculture is to produce plants with agronomically important traits.

Recent advances in genetic manipulation provide the tools to transform plants to contain and express foreign genes. This has led to the development of plants capable of expressing pharmaceuticals and other chemicals, plants with increased pest resistance, increased stress tolerance and many other beneficial traits.

It is often desirable to control expression of a polynucleotide of interest, in a particular tissue, at a particular developmental stage, or under particular conditions, in which the polynucleotide is not normally expressed. The polynucleotide of interest may encode a protein or alternatively may be intended to effect silencing of a corresponding target gene.

Plant promoter sequences are useful in genetic manipulation for directing such spatial, temporal and inducible expression of polynucleotides in transgenic plants. To achieve this, a genetic construct is often introduced into a plant cell or plant. Typically such constructs include a plant promoter operably linked to the polynucleotide sequence of interest. Such a promoter need not normally be associated with the gene of interest. Once transformed, the promoter controls expression of the operably linked polynucleotide of interest thus leading to the desired transgene expression and resulting desired phenotypic characteristics in the plant.

Promoters used in genetic manipulation are typically derived from the 5′ un-transcribed region of genes and contain regulatory elements that are necessary to control expression of the operably linked polynucleotide. Promoters useful for plant biotechnology can be classified depending on when and where they direct expression. For example promoters may be tissue specific or constitutive (capable of transcribing sequences in multiple tissues). Other classes of promoters include inducible promoters that can be triggered on external stimuli such as environmental, and chemical stimuli.

It would be beneficial to have a variety of promoters available in order to ensure that transgenes are transcribed efficiently in the right tissues, at an appropriate stage of growth or development. Additionally it may be desirable to direct a gene expression in response to certain environmental or chemicals signals.

Perennial ryegrass (Lolium perenne L) is the major grass species grown in New Zealand and other temperate climates throughout the world. Valuable traits that may be improved by genetic manipulation of perennial ryegrass include stress tolerance, disease tolerance and nutritional quality. Genetic manipulation of such traits in perennial ryegrass is limited by the availability of promoters capable of appropriately controlling the expression of genes of interest.

It is therefore an object of the present invention to provide a promoter useful for controlling expression of genes in ryegrass and other plants or at least to provide a useful choice.

### SUMMARY OF THE INVENTION

In one aspect the invention provides an isolated promoter polynucleotide comprising at least one of:
a) the sequence of SEQ ID NO:1;
b) a variant of the sequence of SEQ ID NO:1;
c) a fragment of the sequence of SEQ ID NO:1;
d) a fragment of the sequence of b);
e) a variant of the sequence of c); and
f) the complement of any one of a) to e)
   wherein the promoter polynucleotide is capable of controlling transcription of an operably linked polynucleotide in a plant.

In one embodiment the isolated promoter polynucleotide comprises at least one of: 1a) the sequence of SEQ ID NO:1;
b) a sequence with at least 70% identity to the sequence of SEQ ID NO:1;
c) a sequence fragment consisting of at least 50 contiguous nucleotides of the sequence of SEQ ID NO:1;
d) a sequence fragment consisting of at least 50 contiguous nucleotides of the sequence of b);
e) a sequence with at least 70% identity to the sequence of c); and
f) a sequence that is a complement of any one of a) to e) wherein the promoter polynucleotide is capable of controlling transcription of an operably linked polynucleotide in a plant.

In one embodiment, the sequence fragment consists of a sequence selected from any one of SEQ ID NO:10 to SEQ ID NO:16.

Preferably the sequence fragment consists of a sequence selected from any one of SEQ ID NO:10, SEQ ID NO:13 and SEQ ID NO:16.

In one embodiment the promoter polynucleotide is capable of controlling transcription of an operably linked polynucleotide sequence in at least one of leaves, internodes, roots and flowers.

In a further embodiment the promoter polynucleotide is capable of controlling transcription of an operably linked polynucleotide sequence in leaves.

In a further embodiment the promoter polynucleotide is capable of controlling transcription of an operably linked polynucleotide sequence in internodes.

In a further embodiment the promoter polynucleotide is capable of controlling transcription of an operably linked polynucleotide sequence in roots.

In a further embodiment the promoter polynucleotide is capable of controlling transcription of an operably linked polynucleotide sequence in flowers.

Preferably the promoter polynucleotide is capable of controlling transcription of an operably linked polynucleotide sequence in all of leaves, internodes, roots and flowers.

Preferably the promoter polynucleotide is capable of controlling transcription of an operably linked polynucleotide sequence constitutively in substantially all tissues of a plant.

In a further aspect the invention provides a genetic construct comprising a promoter polynucleotide of the invention.

In one embodiment the promoter polynucleotide is operably linked to a polynucleotide sequence to be expressed.

In a further aspect the invention provides a vector comprising a genetic construct of the invention.

In a further aspect the invention provides a plant cell or plant transformed with the promoter polynucleotide of the invention.

In a further aspect the invention provides a plant cell or plant transformed with a genetic construct of the invention.

In a further aspect the invention provides a method for modifying expression of at least one polynucleotide in a plant cell or plant, the method comprising transformation of the plant cell or plant with a promoter polynucleotide of the invention

In a further aspect the invention provides a method for modifying expression of at least one polynucleotide in a plant cell or plant, the method comprising transformation of the plant cell or plant with a genetic construct of the invention.

In a further aspect the invention provides a method for producing a plant cell or plant with modified expression of at least one polynucleotide, the method comprising:
(a) transforming plant cell or plant with a promoter polynucleotide of the invention, and
(b) the cultivating the transgenic plant cell or plant under conditions conducive for the promoter polynucleotide to drive transcription.

In a further aspect the invention provides a method for producing a plant cell or plant with modified expression of at least one polynucleotide, the method comprising:
(a) transforming plant cell or plant with a genetic construct of the invention, and
(b) the cultivating the transgenic plant cell or plant under conditions conducive for the promoter polynucleotide to drive transcription.

In a further aspect the invention provides a method for producing a plant cell or plant with modified expression of at least one gene, the method comprising:
(a) transforming plant cell or plant with a genetic construct of the invention wherein the genetic construct comprises a promoter polynucleotide of the invention operably linked to a polynucleotide sequence to be expressed, and
(b) the cultivating the transgenic plant cell or plant under conditions conducive for the promoter polynucleotide to drive transcription of operably linked sequence.

It will be appreciated by those skilled in the art that, the promoter polynucleotide of the invention may be transformed into the plant to control expression of a polynucleotide operably linked to the promoter prior to transformation. Alternatively the promoter polynucleotide may be transformed into the genome of the plant without an operably linked polynucleotide, but the promoter may control expression of an endogenous polynucleotide, adjacent to the insert site, and typically, to the 3′ end of the inserted promoter polynucleotide.

In a further aspect of the invention provides a method for producing a plant cell or plant with a modified phenotype, the method including the stable incorporation into the genome of the plant, a promoter polynucleotide of the invention

In a further aspect of the invention provides a method for producing a plant cell or plant with a modified phenotype, the method including the stable incorporation into the genome of the plant, a genetic construct of the invention

In a further aspect the invention provides a plant cell or plant produced by a method of the invention.

In a further aspect the invention provides a seed, propagule, progeny or part of a plant, of the invention.

The promoter polynucleotide of the invention may be derived from any species and/or may be produced synthetically or recombinantly.

In one embodiment the promoter polynucleotide, is derived from a plant species.

In a further embodiment the promoter polynucleotide, is derived from a gymnosperm plant species.

In a further embodiment the promoter polynucleotide, is derived from an angiosperm plant species.

In a further embodiment the promoter polynucleotide, is derived from a from dicotyledonous plant species.

In a further embodiment the promoter polynucleotide, is derived from a monocotyledonous plant species.

The polypeptide encoded by the polynucleotide to be expressed in the construct of the invention, may be derived from any species and/or may be produced synthetically or recombinantly.

In one embodiment the polypeptide is derived from a plant species.

In a further embodiment the polypeptide is derived from a gymnosperm plant species.

In a further embodiment the polypeptide is derived from an angiosperm plant species.

In a further embodiment the polypeptide is derived from a from dicotyledonous plant species.

In a further embodiment the polypeptide is derived from a monocotyledonous plant species.

The plant cells and plants, of the invention may be derived from any species.

In one embodiment the plant cell or plant, is derived from a gymnosperm plant species.

In a further embodiment the plant cell or plant, is derived from an angiosperm plant species.

In a further embodiment the plant cell or plant, is derived from a from dicotyledonous plant species.

In a further embodiment the plant cell or plant, is derived from a monocotyledonous plant species.

Preferred dicotyledonous plant genera include: Amygdalus, Anacardium, Anemone, Arachis, Brassica, Cajanus, Cannabis, Carthamus, Carya, Ceiba, Cicer, Claytonia, Coriandrum, Coronilla, Corydalis, Crotalaria, Cyclamen, Dentaria, Dicentra, Dolichos, Eranthis, Glycine, Gossypium, Helianthus, Lathyrus, Lens, Lespedeza, Linum, Lotus, Lupinus, Macadamia, Medicago, Melilotus, Mucuna, Olea, Onobrychis, Ornithopus, Oxalis, Papaver, Phaseolus, Phoenix, Pistacia, Pisum, Prunus, Pueraria, Ribes, Ricinus, Sesamum, Thalictrum, Theobroma, Trifolium, Trigonella, Vicia and Vigna.

Preferred dicotyledonous plant species include: Amygdalus communis, Anacardium occidentale, Anemone americana, Anemone occidentalis, Arachis hypogaea, Arachis hypogea, Brassica napus Rape, Brassica nigra, Brassica campestris, Cajanus cajan, Cajanus indicus, Cannabis sativa, Carthamus tinctorius, Carya illinoinensis, Ceiba pentandra, Cicer arietinum, Claytonia exigua, Claytonia megarhiza, Coriandrum sativum, Coronilla varia, Corydalis flavula, Corydalis sempervirens, Crotalaria juncea, Cyclamen coum, Dentaria laciniata, Dicentra eximia, Dicentra formosa, Dolichos lablab, Eranthis hyemalis, Gossypium arboreum, Gossypium nanking, Gossypium barbadense, Gossypium herbaceum, Gossypium hirsutum, Glycine max, Glycine ussuriensis, Glycine gracilis, Helianthus annus, Lupinus angustifolius, Lupinus luteus, Lupinus mutabilis, Lespedeza sericea, Lespedeza striata, Lotus uliginosus, Lathyrus sativus, Lens culinaris, Lespedeza stipulacea, Linum usitatissimum, Lotus corniculatus, Lupinus albus, Medicago arborea, Medicago falcate, Medicago hispida, Medicago officinalis, Medicago sativa (alfalfa), Medicago tribuloides, Macadamia integrifolia, Medicago arabica, Melilotus albus, Mucuna pruriens, Olea europaea, Onobrychis viciifolia, Ornithopus sativus, Oxalis tuberosa, Phaseolus aureus, Prunus cerasifera, Prunus cerasus, Phaseolus coccineus, Prunus domestica, Phaseolus lunatus, Prunus maheleb, Phaseolus mungo, Prunus persica, Prunus pseudocerasus, Phaseolus vulgaris, Papaver somniferum, Phaseolus acutifolius, Phoenix dactylifera, Pistacia vera, Pisum sativum, Prunus amygdalus, Prunus armeniaca, Pueraria thunbergiana, Ribes nigrum, Ribes rubrum, Ribes grossularia, Ricinus communis, Sesamum indicum, Thalictrum dioicum, Thalictrum flavum, Thalictrum thalictroides, Theobroma cacao, Trifolium augustifolium, Trifolium diffusum, Trifolium hybridum, Trifolium incarnatum, Trifolium ingrescens, Trifolium pratense, Trifolium repens, Trifolium resupinatum, Trifolium subterraneum, Trifolium alexandrinum, Trigonella foenumgraecum, Vicia angustifolia, Vicia atropurpurea, Vicia calcarata, Vicia dasycarpa, Vicia ervilia, Vaccinium oxycoccos, Vicia pannonica, Vigna sesquipedalis, Vigna sinensis, Vicia villosa, Vicia faba, Vicia sative and Vigna angularis.

Preferred monocotyledonous plant genera include: Agropyron, Allium, Alopecurus, Andropogon, Arrhenatherum, Asparagus, Avena, Bambusa, Bellavalia, Brimeura, Brodiaea, Bulbocodium, Bothrichloa, Bouteloua, Bromus, Calamovilfa, Camassia, Cenchrus, Chionodoxa, Chloris, Colchicum, Crocus, Cymbopogon, Cynodon, Cypripedium, Dactylis, Dichanthium, Digitaria, Elaeis, Eleusine, Eragrostis, Eremurus, Erythronium, Fagopyrum, Festuca, Fritillaria, Galanthus, Helianthus, Hordeum, Hyacinthus, Hyacinthoides, Ipheion, Iris, Leucojum, Liatris, Lolium, Lycoris, Miscanthis, Miscanthus×giganteus, Muscari, Ornithogalum, Oryza, Panicum, Paspalum, Pennisetum, Phalaris, Phleum, Poa, Puschkinia, Saccharum, Secale, Setaria, Sorghastrum, Sorghum, Triticum, Vanilla, X Triticosecale Triticale and Zea.

Preferred monocotyledonous plant species include: Agropyron cristatum, Agropyron desertorum, Agropyron elongatum, Agropyron intermedium, Agropyron smithii, Agropyron spicatum, Agropyron trachycaulum, Agropyron trichophorum, Allium ascalonicum, Allium cepa, Allium chinense, Allium porrum, Allium schoenoprasum, Allium fistulosum, Allium sativum, Alopecurus pratensis, Andropogon gerardi, Andropogon gerardii, Andropogon scoparious, Arrhenatherum elatius, Asparagus officinalis, Avena nuda, Avena sativa, Bambusa vulgaris, Bellevalia trifoliate, Brimeura amethystina, Brodiaea californica, Brodiaea coronaria, Brodiaea elegans, Bulbocodium versicolor, Bothrichloa barbinodis, Bothrichloa ischaemum, Bothrichloa saccharoides, Bouteloua curipendula, Bouteloua eriopoda, Bouteloua gracilis, Bromus erectus, Bromus inermis, Bromus riparius, Calamovilfa longifilia, Camassia scilloides, Cenchrus ciliaris, Chionodoxa forbesii, Chloris gayana, Colchicum autumnale, Crocus sativus, Cymbopogon nardus, Cynodon dactylon, Cypripedium acaule, Dactylis glomerata, Dichanthium annulatum, Dichanthium aristatum, Dichanthium sericeum, Digitaria decumbens, Digitaria smutsii, Elaeis guineensis, Elaeis oleifera, Eleusine coracan, Elymus angustus, Elymus junceus, Eragrostis curvula, Eragrostis tef, Eremurus robustus, Erythronium elegans, Erythronium helenae, Fagopyrum esculentum, Fagopyrum tataricum, Festuca arundinacea, Festuca ovina, Festuca pratensis, Festuca rubra, Fritillaria cirrhosa, Galanthus nivalis, Helianthus annuus sunflower, Hordeum distichum, Hordeum vulgare, Hyacinthus orientalis, Hyacinthoides hispanica, Hyacinthoides non-scripta, Ipheion sessile, Iris collettii, Iris danfordiae, Iris reticulate, Leucojum aestivum, Liatris cylindracea, Liatris elegans, Lilium longiflorum, Lolium multiflorum, Lolium perenne, Lycoris radiata, Miscanthis sinensis, Miscanthus×giganteus, Muscari armeniacum, Muscari macrocarpum, Narcissus pseudonarcissus, Ornithogalum montanum, Oryza sativa, Panicum italicium, Panicum maximum, Panicum miliaceum, Panicum purpurascens, Panicum virgatum, Panicum virgatum, Paspalum dilatatum, Paspalum notatum, Pennisetum clandestinum, Pennisetum glaucum, Pennisetum purpureum, Pennisetum spicatum, Phalaris arundinacea, Phleum bertolinii, Phleum pratense, Poa fendleriana, Poa pratensis, Poa nemoralis, Puschkinia scilloides, Saccharum offlcinarum, Saccharum robustum, Saccharum sinense, Saccharum spontaneum, Scilla autumnalis, Scilla peruviana, Secale cereale, Setaria italica, Setaria sphacelata, Sorghastrum nutans, Sorghum bicolor, Sorghum dochna, Sorghum halepense, Sorghum sudanense, Trillium grandiflorum, Triticum aestivum, Triticum dicoccum, Triticum durum, Triticum monococcum, Tulipa batalinii, Tulipa clusiana, Tulipa dasystemon, Tulipa gesneriana, Tulipa greigii, Tulipa kaufmanniana, Tulipa sylvestris, Tulipa turkestanica, Vanilla fragrans, X Triticosecale and Zea mays.

Other preferred plants are forage plants from a group comprising but not limited to the following genera: Lolium, Festuca, Dactylis, Bromus, Trifolium, Medicago, Phleum, Phalaris, Holcus, Lotus, Plantago and Cichorium.

Particularly preferred forage plants are from the genera Lolium and Trifolium. Particularly preferred species are Lolium perenne and Trifolium repens.

Particularly preferred monocotyledonous plant species are: Lolium perenne and Oryza sativa.

A particularly preferred plant species is Lolium perenne.

### DETAILED DESCRIPTION

The applicants have identified a promoter polynucleotide sequence from perennial ryegrass (Lolium perenne) and demonstrated that the promoter regulates transcription of an operably linked polynucleotide in at least one of leaves, internodes, roots and flowers. The invention also provides variants and fragments of the promoter polynucleotide. The invention provides genetic constructs and vectors comprising the promoter polynucleotide sequences, and transgenic plant cells and transgenic plants comprising the promoter polynucleotide sequence, genetic constructs, or vectors of the invention.

The invention also provides methods for modifying expression of genes in plants and modifying phenotype in plants, and methods for producing plants with modified gene expression and modified phenotype. The invention further provides plants produced by the methods of the invention.

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### Polynucleotides and Fragments

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

A “fragment” of a polynucleotide sequence provided herein is a subsequence of contiguous nucleotides that is preferably at least 15 nucleotides in length. The fragments of the invention preferably comprises at least 20 nucleotides, more preferably at least 30 nucleotides, more preferably at least 40 nucleotides, more preferably at least 50 nucleotides and most preferably at least 60 contiguous nucleotides of a polynucleotide of the invention. A fragment of a polynucleotide sequence can be used in antisense, gene silencing, triple helix or ribozyme technology, or as a primer, a probe, included in a microarray, or used in polynucleotide-based selection methods.

The term “fragment” in relation to promoter polynucleotide sequences is intended to include sequences comprising cis-elements and regions of the promoter polynucleotide sequence capable of regulating expression of a polynucleotide sequence to which the fragment is operably linked.

Preferably fragments of promoter polynucleotide sequences of the invention comprise at least 20, more preferably at least 30, more preferably at least 40, more preferably at least 50, more preferably at least 100, more preferably at least 200, more preferably at least 300, more preferably at least 400, more preferably at least 500, more preferably at least 600, more preferably at least 700, more preferably at least 800, more preferably at least 900 and most preferably at least 1000 contiguous nucleotides of a promoter polynucleotide of the invention.

The term “primer” refers to a short polynucleotide, usually having a free 3′OH group, that is hybridized to a template and used for priming polymerization of a polynucleotide complementary to the template. Such a primer is preferably at least 5, more preferably at least 6, more preferably at least 7, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20 nucleotides in length.

The term “probe” refers to a short polynucleotide that is used to detect a polynucleotide sequence, that is complementary to the probe, in a hybridization-based assay. The probe may consist of a “fragment” of a polynucleotide as defined herein. Preferably such a probe is at least 5, more preferably at least 10, more preferably at least 20, more preferably at least 30, more preferably at least 40, more preferably at least 50, more preferably at least 100, more preferably at least 200, more preferably at least 300, more preferably at least 400 and most preferably at least 500 nucleotides in length.

The term “derived from” with respect to polynucleotides of the invention being “derived from” a particular genera or species, means that the polynucleotide has the same sequence as a polynucleotide found naturally in that genera or species. The polynucleotide which is derived from a genera or species may therefore be produced synthetically or recombinantly.

### Polypeptides and Fragments

The term “polypeptide”, as used herein, encompasses amino acid chains of any length but preferably at least 5 amino acids, including full-length proteins, in which amino acid residues are linked by covalent peptide bonds. The polypeptides may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof.

A “fragment” of a polypeptide is a subsequence of the polypeptide that performs a function that is required for the biological activity and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof capable of performing the above enzymatic activity.

The term “isolated” as applied to the polynucleotide or polypeptide sequences disclosed herein is used to refer to sequences that are removed from their natural cellular environment. An isolated molecule may be obtained by any method or combination of methods including biochemical, recombinant, and synthetic techniques.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

The term “derived from” with respect to polypeptides disclosed being derived from a particular genera or species, means that the polypeptide has the same sequence as a polypeptide found naturally in that genera or species. The polypeptide, derived from a particular genera or species, may therefore be produced synthetically or recombinantly.

### Variants

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the inventive polynucleotides and polypeptides possess biological activities that are the same or similar to those of the inventive polynucleotides or polypeptides. The term “variant” with reference to polynucleotides and polypeptides encompasses all forms of polynucleotides and polypeptides as defined herein.

### Polynucleotide Variants

Variant polynucleotide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a specified polynucleotide sequence. Identity is found over a comparison window of at least 20 nucleotide positions, more preferably at least 50 nucleotide positions, more preferably at least 100 nucleotide positions, more preferably at least 200 nucleotide positions, more preferably at least 300 nucleotide positions, more preferably at least 400 nucleotide positions, more preferably at least 500 nucleotide positions, more preferably at least 600 nucleotide positions, more preferably at least 700 nucleotide positions, more preferably at least 800 nucleotide positions, more preferably at least 900 nucleotide positions, more preferably at least 1000 nucleotide positions and most preferably over the entire length of the specified polynucleotide sequence.

Variant promoter polynucleotides of the invention preferably comprise at least one copy of one, more preferably at least one copy of two, even more preferably at least one copy of three and most preferably at least one copy of four of the four light-inducible promoter motifs: ACTTTG (T-box promoter motif) TGATAA (GATA promoter motif), GCCAC(SORLIP1) and GGGCC (SORLIP2). In addition variant promoter polynucleotides of the invention preferably comprise at least one copy of one, more preferably at least one copy of two, even more preferably at least one copy of three and most preferably at least one copy of four of the following cis-element sequences TGTCTC (ARF binding site), TTTGACC (W-box promoter motif), CATGCATG (RY repeat motif) and AACCCC (PAL promoter motif).

Polynucleotide sequence identity can be determined in the following manner. The subject polynucleotide sequence is compared to a candidate polynucleotide sequence using BLASTN (from the BLAST suite of programs, version 2.2.5 [November 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI on the worldwide web at ftp://ftp.ncbi.nih.gov/blast/. The default parameters of bl2seq are utilized except that filtering of low complexity parts should be turned off.

The identity of polynucleotide sequences may be examined using the following unix command line parameters:

bl2seq −i nucleotideseq1 −j nucleotideseq2 −F F −p blastn

The parameter −F F turns off filtering of low complexity sections. The parameter −p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained on the worldwide web at http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program, which computes an optimal global alignment of two sequences without penalizing terminal gaps, may be used to calculate sequence identity. GAP is described in the following paper: Huang, X. (1994) On Global Sequence Alignment. Computer Applications in the Biosciences 10, 227-235.

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polynucleotides may be determined using the publicly available bl2seq program from the BLAST suite of programs (version 2.2.5 [November 2002]) available from NCBI on the worldwide web at ftp://ftp.ncbi.nih.gov/blast/.

The similarity of polynucleotide sequences may be examined using the following unix command line parameters:

bl2seq −i nucleotideseq1 −j nucleotideseq2 −F F −p tblastx

The parameter −F F turns off filtering of low complexity sections. The parameter −p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10⁻¹⁰ more preferably less than 1×10⁻²⁰, more preferably less than 1×10⁻³⁰, more preferably less than 1×10⁻⁴⁰, more preferably less than 1×10⁻⁵⁰, more preferably less than 1×10⁻⁶⁰, more preferably less than 1×10⁻⁷⁰, more preferably less than 1×10⁻⁸⁰, more preferably less than 1×10⁻⁹⁰ and most preferably less than 1×10⁻¹°° when compared with any one of the specifically identified sequences.

Alternatively, variant polynucleotides of the present invention hybridize to a specified polynucleotide sequence, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81. 5+0.41% (G+C-log(Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in lx SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 by is reduced by approximately (500/oligonucleotide length)° C.

With respect to the DNA mimics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

Variant polynucleotides such as those in constructs of the invention encoding stress-protective protein, also encompasses polynucleotides that differ from the specified sequences but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also contemplated. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available bl2seq program from the BLAST suite of programs (version 2.2.5 [November 2002]), available from NCBI on the worldwide web at ftp://ftp.ncbi.nih.gov/blast/, via the tblastx algorithm as previously described.

### Constructs, Vectors and Components Thereof.

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain a promoter polynucleotide such as a promoter polynucleotide of the invention including the necessary elements that permit transcribing the insert polynucleotide molecule, and, optionally, translating the transcript into a polypeptide. The insert polynucleotide molecule may be derived from the host cell, or may be derived from a different cell or organism and/or may be a synthetic or recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
a) a promoter, such as a promoter polynucleotide sequence of the invention, functional in the host cell into which the construct will be transformed,
b) the polynucleotide to be expressed, and
c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

“Operably-linked” means that the sequenced to be expressed is placed under the control of regulatory elements that include promoters, tissue-specific regulatory elements, temporal regulatory elements, enhancers, repressors and terminators.

The term “noncoding region” includes to untranslated sequences that are upstream of the translational start site and downstream of the translational stop site. These sequences are also referred to respectively as the 5′ UTR and the 3′ UTR. These sequences may include elements required for transcription initiation and termination and for regulation of translation efficiency. The term “noncoding” also includes intronic sequences within genomic clones.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to a polynucleotide sequence capable of regulating the expression of a polynucleotide sequence to which the promoter is operably linked. Promoters may comprise cis-initiator elements which specify the transcription initiation site and conserved boxes such as the TATA box, and motifs that are bound by transcription factors.

### Methods for Isolating or Producing Polynucleotides

The polynucleotide molecules of the invention can be isolated by using a variety of techniques known to those of ordinary skill in the art. By way of example, such polynucleotides can be isolated through use of the polymerase chain reaction (PCR) described in Mullis et al., Eds. 1994 The Polymerase Chain Reaction, Birkhauser, incorporated herein by reference. The polynucleotides of the invention can be amplified using primers, as defined herein, derived from the polynucleotide sequences of the invention.

Further methods for isolating polynucleotides of the invention, or useful in the methods of the invention, include use of all or portions, of the polynucleotides set forth herein as hybridization probes. The technique of hybridizing labeled polynucleotide probes to polynucleotides immobilized on solid supports such as nitrocellulose filters or nylon membranes, can be used to screen the genomic or cDNA libraries. Exemplary hybridization and wash conditions are: hybridization for 20 hours at 65° C. in 5.0×SSC, 0.5% sodium dodecyl sulfate, 1× Denhardt's solution; washing (three washes of twenty minutes each at 55° C.) in 1.0×SSC, 1% (w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

The polynucleotide fragments of the invention may be produced by techniques well-known in the art such as restriction endonuclease digestion, oligonucleotide synthesis and PCR amplification.

A partial polynucleotide sequence may be used, in methods well-known in the art to identify the corresponding full length polynucleotide sequence. Such methods include PCR-based methods, 5′RACE (Frohman M A, 1993, Methods Enzymol. 218: 340-56) and hybridization-based method, computer/database-based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a polynucleotide. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

It may be beneficial, when producing a transgenic plant from a particular species, to transform such a plant with a sequence or sequences derived from that species. The benefit may be to alleviate public concerns regarding cross-species transformation in generating transgenic organisms. Additionally when down-regulation of a gene is the desired result, it may be necessary to utilise a sequence identical (or at least highly similar) to that in the plant, for which reduced expression is desired. For these reasons among others, it is desirable to be able to identify and isolate orthologues of a particular gene in several different plant species. Variants (including orthologues) may be identified by the methods described.

The promoter sequences disclosed may be characterized to identify fragments, such as cis-elements and regions, capable of regulating to expression of operably linked sequences, using techniques well-known to those skilled in the art. Such techniques include 5′ and/or 3′ deletion analysis, linker scanning analysis and various DNA footprinting techniques (Degenhardt et al., 1994 Plant Cell:6(8) 1123-34; Directed Mutagenesis: A Practical Approach IRL Press (1991)). Fragments include truncated versions of longer promoter sequences which may terminate (at the 3′ end) at or close to the transcriptional start site. Methods for identifying the transcription start site of a promoter are well-known to those skilled in the art (discussed in Hashimoto et al., 2004, Nature Biotechnology 22, 1146-1149).

The techniques described above may be used to identify a fragment that defines essential region of the promoter that is able to confer the desired expression profile. The essential region may function by itself or may be fused to a core promoter to drive expression of an operably linked polynucleotide.

The core promoter can be any one of known core promoters such as the Cauliflower Mosaic Virus 35S or 19S promoter (U.S. Pat. No. 5,352,605), ubiquitin promoter (U.S. Pat. No. 5,510,474) the IN2 core promoter (U.S. Pat. No. 5,364,780) or a Figwort Mosaic Virus promoter (Gruber, et al. “Vectors for Plant Transformation” Methods in Plant Molecular Biology and Biotechnology) et al. eds, CRC Press pp. 89-119 (1993)).

Promoter fragments can be tested for their utility in driving expression in any particular cell or tissue type, or at any particular developmental stage, or in response to any particular stimulus by techniques well-known to those skilled in the art. Techniques include operably-linking the promoter fragment to a reporter or other polynucleotide and measuring report activity or polynucleotide expressions in plants in response to stress. Such techniques are described in the Examples section of this specification.

### Methods for Identifying Variants

### Physical Methods

Variant polynucleotides may be identified using PCR-based methods (Mullis et al., Eds. 1994 The Polymerase Chain Reaction, Birkhauser).

Alternatively library screening methods, well known to those skilled in the art, may be employed (Sambrook et al., Molecular Cloning: A Laboratory Manual, 2nd Ed. Cold Spring Harbor Press, 1987). When identifying variants of the probe sequence, hybridization and/or wash stringency will typically be reduced relatively to when exact sequence matches are sought.

### Computer-Based Methods

Polynucleotide and polypeptide variants, may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29: 1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [November 2002]) including BLASTN, BLASTP, BLASTX, tBLASTN and tBLASTX, which are publicly available on the worldwide web at ftp://ftp.ncbi.nih.goviblast/ or from the National Center for Biotechnology Information (NCBI), National Library of Medicine, Building 38A, Room 8N805, Bethesda, Md. 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, including BLASTN, BLASTP, and BLASTX, is described in the publication of Altschul et al., Nucleic Acids Res. 25: 3389-3402, 1997.

The “hits” to one or more database sequences by a queried sequence produced by BLASTN, BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, on the worldwide web at http://www-igbmc.u-strasbg.fr/BioInfo/ClustalW/Top.html) or T-COFFEE (Cedric Notredame, Desmond G. Higgins, Jaap Hering a, T-Coffee: A novel method for fast and accurate multiple sequence alignment, J. Mol. Biol. (2000) 302: 205-217) or PILEUP, which uses progressive, pairwise alignments. (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

### Methods for Producing Constructs and Vectors

The genetic constructs of the present invention comprise one or more polynucleotide sequences of the invention and/or polynucleotides encoding polypeptides disclosed, and may be useful for transforming, for example, bacterial, fungal, insect, mammalian or particularly plant organisms. The genetic constructs of the invention are intended to include expression constructs as herein defined.

Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

### Methods for Producing Host Cells Comprising Constructs and Vectors

The invention provides a host cell which comprises a genetic construct or vector of the invention. Host cells may be derived from, for example, bacterial, fungal, insect, mammalian or plant organisms.

Host cells comprising genetic constructs, such as expression constructs, of the invention are useful in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides. Such methods may involve the culture of host cells in an appropriate medium in conditions suitable for or conducive to expression of a polypeptide of the invention. The expressed recombinant polypeptide, which may optionally be secreted into the culture, may then be separated from the medium, host cells or culture medium by methods well known in the art (e.g. Deutscher, Ed, 1990, Methods in Enzymology, Vol 182, Guide to Protein Purification).

### Methods for Producing Plant Cells and Plants Comprising Constructs and Vectors

The invention further provides plant cells which comprise a genetic construct of the invention, and plant cells modified to alter expression of a polynucleotide or polypeptide. Plants comprising such cells also form an aspect of the invention.

Methods for transforming plant cells, plants and portions thereof with polynucleotides are described in Draper et al., 1988, Plant Genetic Transformation and Gene Expression. A Laboratory Manual, Blackwell Sci. Pub. Oxford, p. 365; Potrykus and Spangenburg, 1995, Gene Transfer to Plants. Springer-Verlag, Berlin.; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

The following are representative publications disclosing genetic transformation protocols that can be used to genetically transform the following plant species: Rice (Alam et al., 1999, Plant Cell Rep. 18, 572); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9, 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. No. 5,846,797 and 5,004,863); perennial ryegrass (Bajaj et al., 2006, Plant Cell Rep. 25, 651); grasses (U.S. Pat. Nos. 5,187,073, 6,020,539); peppermint (Niu et al., 1998, Plant Cell Rep. 17, 165); citrus plants (Pena et al., 1995, Plant Sci. 104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); brassica (U.S. Pat. Nos. 5,188,958; 5,463,174 and 5,750,871); and cereals (U.S. Pat. No. 6,074,877). Other species are contemplated and suitable methods and protocols are available in the scientific literature for use by those skilled in the art.

### Methods for Genetic Manipulation of Plants

A number of strategies for genetically manipulating plants are available (e.g. Birch, 1997, Ann Rev Plant Phys Plant Mol Biol, 48, 297). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. Strategies may also be designed to increase expression of a polynucleotide/polypeptide in response to an external stimuli, such as an environmental stimuli. Environmental stimuli may include environmental stresses such as mechanical (such as herbivore activity), dehydration, salinity and temperature stresses. The expressed polynucleotide/polypeptide may be derived from the plant species to be transformed or may be derived from a different plant species.

Transformation strategies may be designed to reduce expression of a polynucleotide/polypeptide in a plant cell, tissue, organ or at a particular developmental stage which/when it is normally expressed or to reduce expression of a polynucleotide/polypeptide in response to an external stimuli. Such strategies are known as gene silencing strategies.

Genetic constructs for expression of genes in transgenic plants typically include promoters, such as promoter polynucleotides of the invention, for driving the expression of one or more cloned polynucleotide, terminators and selectable marker sequences to detect presence of the genetic construct in the transformed plant.

Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zin gene terminator, the Oryza sativa ADP-glucose pyrophosphorylase terminator and the Solanum tuberosum PI-II terminator.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Use of genetic constructs comprising reporter genes (coding sequences which express an activity that is foreign to the host, usually an enzymatic activity and/or a visible signal (e.g., luciferase, GUS, GFP) which may be used for promoter expression analysis in plants and plant tissues are also contemplated. The reporter gene literature is reviewed in Herrera-Estrella et al., 1993, Nature 303, 209, and Schrott, 1995, In: Gene Transfer to Plants (Potrykus, T., Spangenbert. Eds) Springer Verlag. Berline, pp. 325-336.

Gene silencing strategies may be focused on the gene itself or regulatory elements which effect expression of the encoded polypeptide. “Regulatory elements” is used here in the widest possible sense and includes other genes which interact with the gene of interest.

Genetic constructs designed to decrease or silence the expression of a polynucleotide/polypeptide may include an antisense copy of a polynucleotide. In such constructs the polynucleotide is placed in an antisense orientation with respect to the promoter and terminator.

An “antisense” polynucleotide is obtained by inverting a polynucleotide or a segment of the polynucleotide so that the transcript produced will be complementary to the mRNA transcript of the gene, e.g.,

| | |
|---|---|
| 5′GATCTA 3′ | 3′CTAGAT 5′ |
| (coding strand) | (antisense strand) |
| 3′CUAGAU 5′ | 5′GAUCUCG 3′ |
| mRNA | antisense RNA |

Genetic constructs designed for gene silencing may also include an inverted repeat. An ‘inverted repeat’ is a sequence that is repeated where the second half of the repeat is in the complementary strand, e.g.,

5′-GATCTA . . . TAGATC-3′
3′-CTAGAT . . . ATCTAG-5′

The transcript formed may undergo complementary base pairing to form a hairpin structure. Usually a spacer of at least 3-5 by between the repeated region is required to allow hairpin formation.

Another silencing approach involves the use of a small antisense RNA targeted to the transcript equivalent to an miRNA (Llave et al., 2002, Science 297, 2053). Use of such small antisense RNA corresponding to polynucleotide of the invention is expressly contemplated.

The term genetic construct as used herein also includes small antisense RNAs and other such polypeptides effecting gene silencing.

Transformation with an expression construct, as herein defined, may also result in gene silencing through a process known as sense suppression (e.g. Napoli et al., 1990, Plant Cell 2, 279; de Carvalho Niebel et al., 1995, Plant Cell, 7, 347). In some cases sense suppression may involve over-expression of the whole or a partial coding sequence but may also involve expression of non-coding region of the gene, such as an intron or a 5′ or 3′ untranslated region (UTR). Chimeric partial sense constructs can be used to coordinately silence multiple genes (Abbott et al., 2002, Plant Physiol. 128(3): 844-53; Jones et al., 1998, Planta 204: 499-505). The use of such sense suppression strategies to silence the expression of a sequence operably-linked to promoter of the invention is also contemplated.

The polynucleotide inserts in genetic constructs designed for gene silencing may correspond to coding sequence and/or non-coding sequence, such as promoter and/or intron and/or 5′ or 3′ UTR sequence, or the corresponding gene.

Other gene silencing strategies include dominant negative approaches and the use of ribozyme constructs (McIntyre, 1996, Transgenic Res, 5, 257)

Pre-transcriptional silencing may be brought about through mutation of the gene itself or its regulatory elements. Such mutations may include point mutations, frameshifts, insertions, deletions and substitutions.

### Plants

The term “plant” is intended to include a whole plant or any part of a plant, propagules and progeny of a plant.

The term ‘propagule’ means any part of a plant that may be used in reproduction or propagation, either sexual or asexual, including seeds and cuttings.

A “transgenic” or transformed” plant refers to a plant which contains new genetic material as a result of genetic manipulation or transformation. The new genetic material may be derived from a plant of the same species as the resulting transgenic of transformed plant or from a different species. A transformed plant includes a plant which is either stably or transiently transformed with new genetic material.

The plants of the invention may be grown and either self-ed or crossed with a different plant strain and the resulting hybrids, with the desired phenotypic characteristics, may be identified. Two or more generations may be grown. Plants resulting from such standard breeding approaches also form an aspect of the present invention.

### BRIEF DESCRIPTION OF THE DRAWINGS

FIG. 1 shows the promoter polynucleotide sequence of SEQ ID NO:1, showing the predicted transcription start site (uppercase T).

FIG. 2 shows results of a Tf sitescan/dynamicPlus (available on the worldwide web at http://www.ifti.org) (Ghosh D. 2000, Nucleic Acids Research 28:308-10) analysis of the promoter of SEQ ID NO:1.

FIG. 3 shows the results of Signal Scan (available on the worldwide web at http://www.dna.affrc.go.jp/PLACE/signalscan.html) analysis of the promoter of SEQ ID NO:1.

FIG. 4 shows a DNA gel-blot analysis of transgenic (TO) rice lines (1104001, 1104003, 113101, 113103, 113104) transformed with the bacterial uidA gene driven by PRO7.

FIG. 5 shows the map of a binary vector including PRO7 operably linked to the bacterial uidA gene.

FIG. 6 shows the results from histochemical examination for bacterial uidA expression by GUS activity analysis in two independent transgenic lines (1113103 and 1113104) of rice.

FIG. 7 shows the map of a binary vector including PRO7ΔPstI operably linked to the bacterial uidA gene.

FIG. 8 shows the map of a binary vector including PRO7ΔPmlI-SpeI operably linked to the bacterial uidA gene.

FIG. 9 shows the map of a binary vector including PRO7ΔPmlI-StuI operably linked to the bacterial uidA gene.

FIG. 10 shows a schematic representation of promoter deletion series with the summary of the GUS assay result. Lower panel shows the results from histochemical examination for bacterial uidA expression by GUS activity analysis in leaves of independent transgenic lines of perennial ryegrass.

FIG. 11 shows relative efficiency in gene expression driven by either CaMVD35S or PRO7 and its deletion fragments as determined by qRT-PCR.

### EXAMPLES

The invention will now be illustrated with reference to the following non-limiting examples.

### Example 1

### Identification and Characterisation of the Ryegrass Promoter Sequence of the Invention

Hypomethylated genomic DNA from Lolium perenne cv. Bronsyn was isolated and sequenced (Orion Genomics, St Louis). A hypomethylated genomic DNA sequence of 1664 by (SEQ ID NO:1) was identified as containing a 5′ transcriptional regulatory region based on the sequence homology to a 5′ CDS. A set of 4 nested primers were designed (Flanking forward primer, SEQ ID NO: 3: CTAGGTCCAGAGTGTAGG; Flanking reverse primer, SEQ ID NO: 4: TTCCACCGCCCGCACTTGAC; Nested forward primer, SEQ ID NO: 5: CTCACACCTAATTGTCCGG; and Nested reverse primer, SEQ ID NO: 6: TCTCTACCTATGCGAGCTAC) to enable us to clone the promoter from the targeted Lolium perenne genomic DNA.

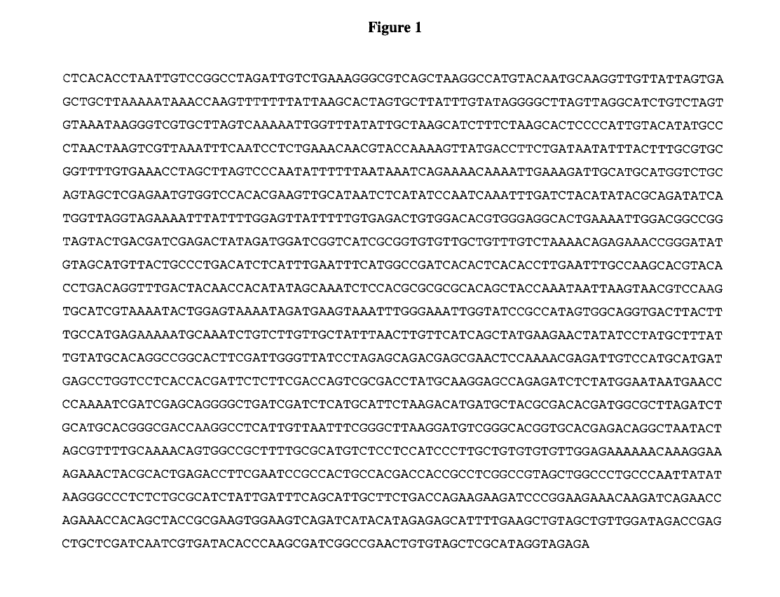

The applicants predicted the transcription start site at base 1467, a thymine base, using tools available on the worldwide web at http://www.fruitfly.org/seq_tools/promoter.html. The result is shown in FIG. 1.

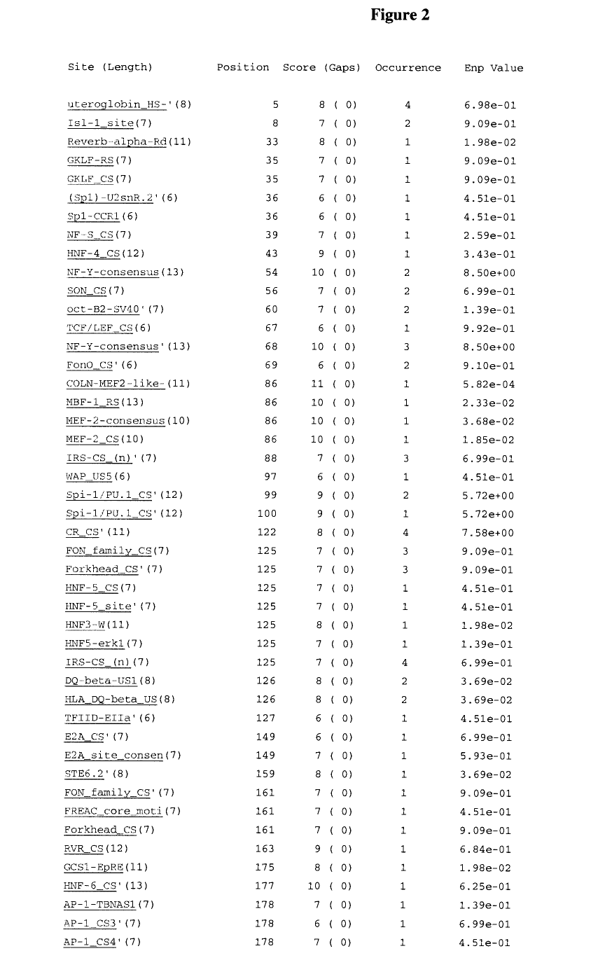

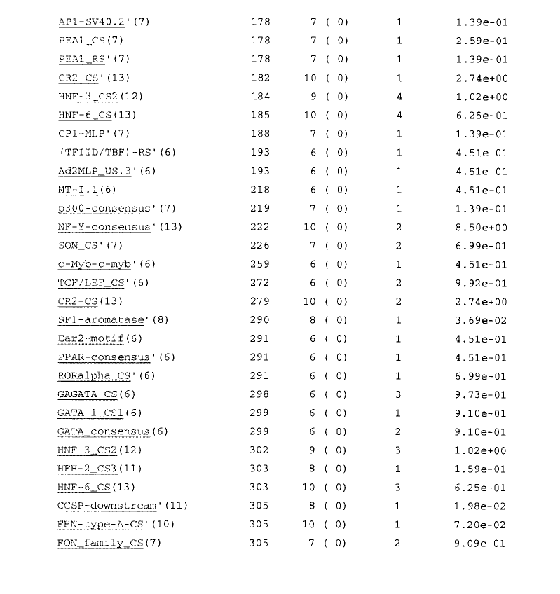

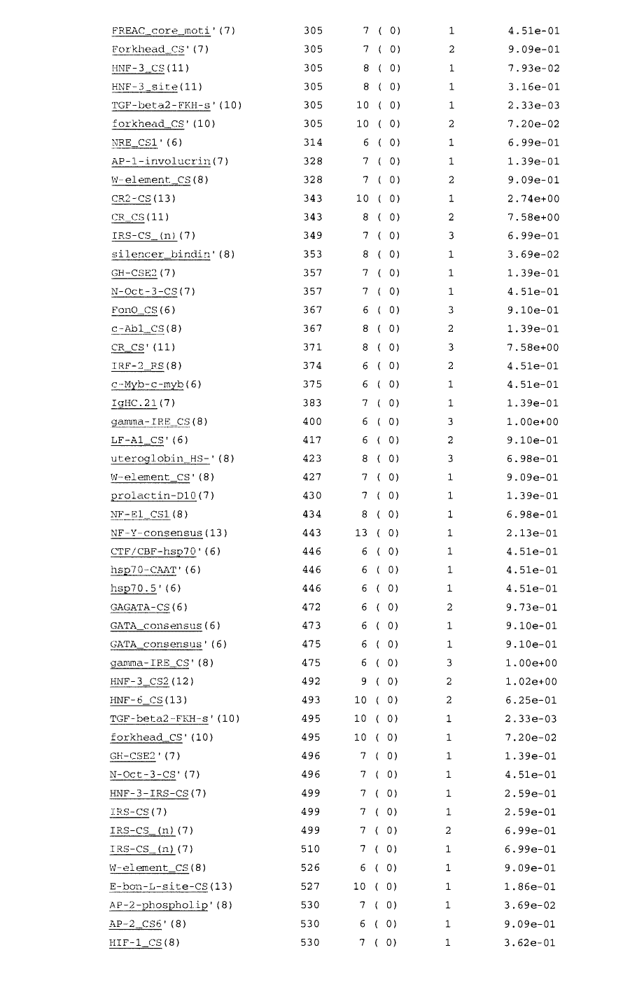

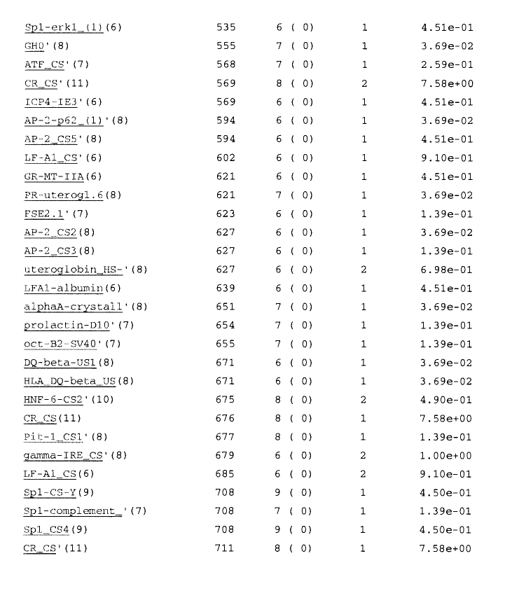

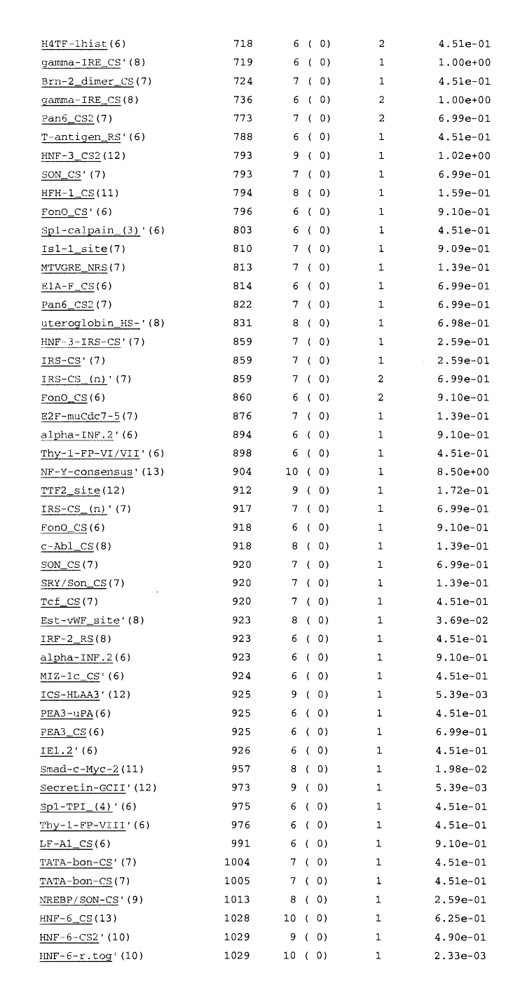

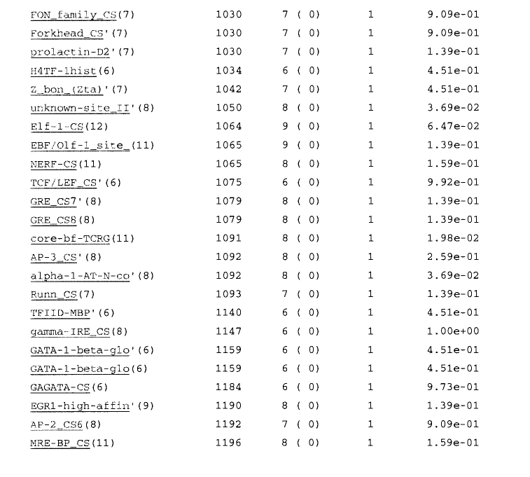

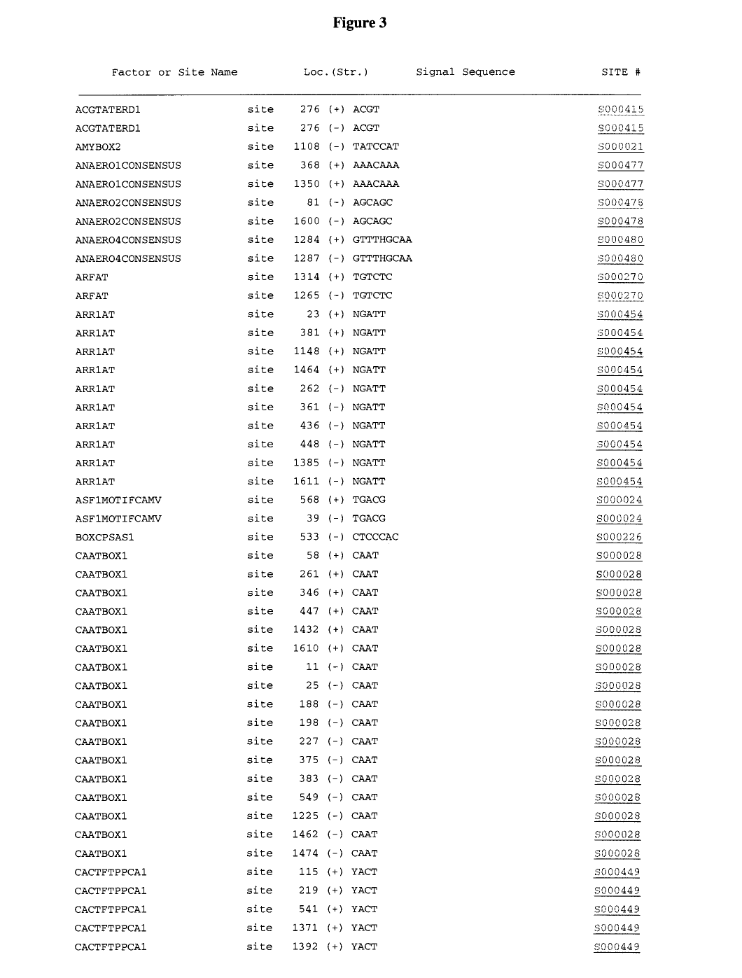

The applicants used both Tf site scan/dynamicPlus (available on the worldwide web at http://www.ifti.org) (Ghosh D. 2000, NAR, 28:308-10) and Signal Scan (available on the worldwide web at http://www.dna.affrc.go.jp/PLACE/signalscan.html) to identify transcription factor binding sites and cis-acting elements in the sequence of SEQ ID NO:1 The results are shown in FIGS. 2 and 3 respectively. Amongst these, the following sites are very likely to have an impact as to the manner in which the promoter functions:

T-box promoter motif (ACTTTG)—light activated
SORLIPI (GCCAC)—light activated
SORLIP2 (GGGCC)—light activated
GATA promoter motif (TGATAA), —light activated
ARF binding site (TGTCTC)—auxin responsive
RY repeat motif (CATGCATG)—proper expression in seeds
W-box promoter motif (TTTGACC)—biotic stress responsive
PAL promoter motif (AACCCC)—wound induced activation.

### Example 2

### Demonstration of Control of Gene Expression by the Ryegrass Promoter of the Invention

### Preparation of a Promoter Reporter Construct

A 1664 by DNA sequence fragment was amplified by PCR from the sequence of SEQ ID NO:1 using two pairs of oligonucleotide sequences (SEQ ID NO: 3 to 6) and inserted into a T-tailed cloning entry vector that enables a transcriptional fusion between the ryegrass promoter and the GUS reporter gene (Jefferson R. A., et al., 1987. EMBO 6:3901-3907). Clones were sequenced and a positive clone was selected based on sequence analysis indicating that the promoter is in the correct orientation to drive the reporter gene. The promoter-reporter and terminator cassette was excised by digesting with the restriction enzyme PacI. The PacI fragment was ligated in the binary vector at the PacI site to result in the PRO7 binary construct. A map of the PRO7 binary construct is shown in FIG. 5. The sequence of the binary construct is shown in SEQ ID NO:2.

Table 1 below shows features of the PRO7 construct.

TABLE 1
Description:

| Type | Start | End | C* | Name | Description |
|---|---|---|---|---|---|
| REGION | 261 | 286 | | RB | |
| REGION | 6520 | 6545 | | LB | |
| REGION | 6810 | 6595 | C | 35S 3′Term | |
| GENE | 7861 | 6839 | C | Hpt | |
| Promoter | 8678 | 7897 | C | ′CaMV35S Pr. | |
| REGION | 9179 | 8937 | C | NOS 3′Term | |
| GENE | 11214 | 9190 | C | uidA | GUS encoding |
| | | | | | gene with intron |
| Promoter | 12895 | 11230 | C | PR07 | |

Note:
C* = Complimentary sequence

### Plant Transformation

Rice (Oryza sativa spp japonica cv. Niponbarre) was transformed using an immature embryo based system (Metahelix Life Sciences, India). Immature panicles, post-milky stage were used to source embryos. Freshly isolated immature embryos were co-cultivated with Agrobacterium tumefaciens (A. tumefaciens) harboring the promoter/GUS binary construct (PRO7) described above for 48-64 h. A. tumefaciens were eliminated by antibiotic treatment and the explants were transferred to selection medium where the transformed plant cells proliferate to give rise to uniformly transformed calli. The selection medium had a combination of 2,4-D and benzylaminopurine. After 3-4 weeks of selection, the calli were transferred to a regeneration medium containing increased cytokinin and decreased auxin concentration relative to the selection medium. Shoot and root were initiated in this medium. Plantlets were transferred to a glasshouse for hardening. Five primary transformed (T₀) plants from five independent transformation events were established in the glasshouse. Twenty seeds each from two of the five Tₒ events were grown to produce T₁ plants, which were phenotyped for GUS expression and activity (Tables 2 and 3; FIG. 6).

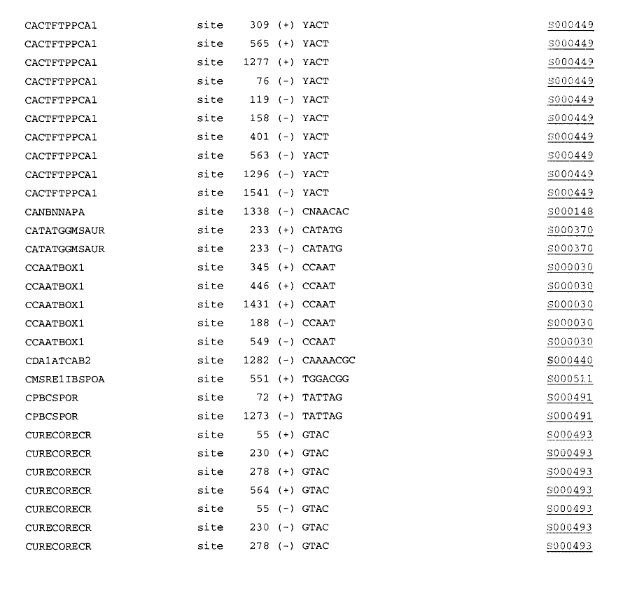

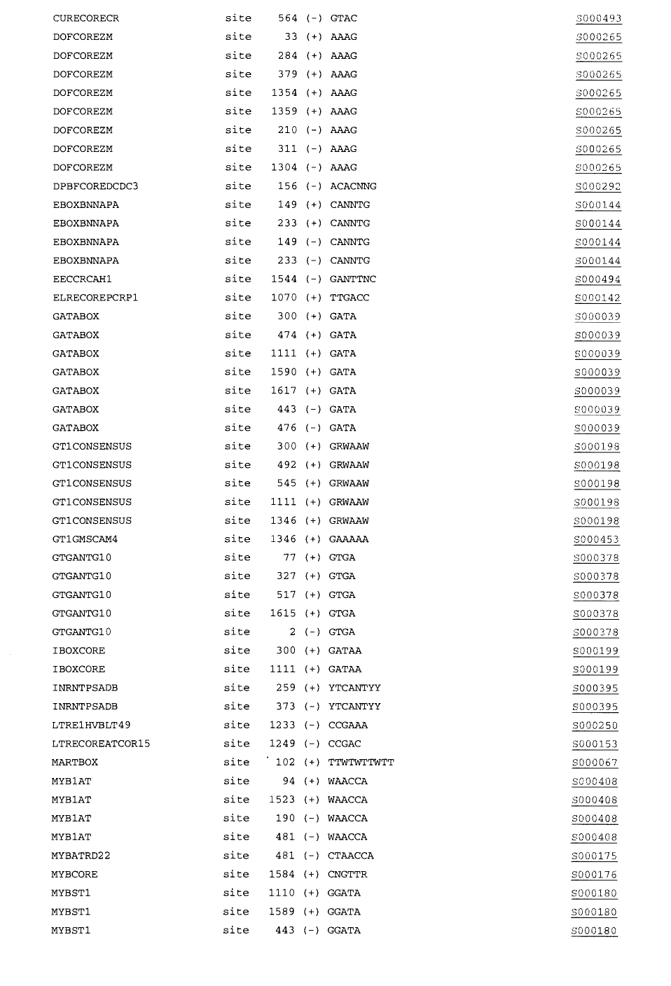

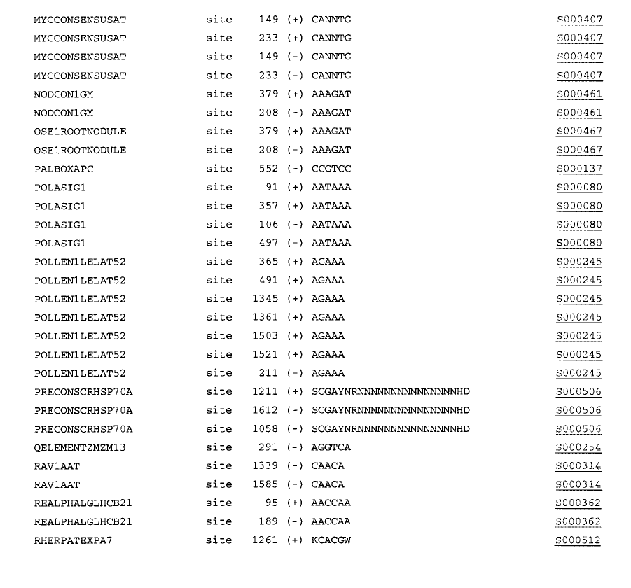

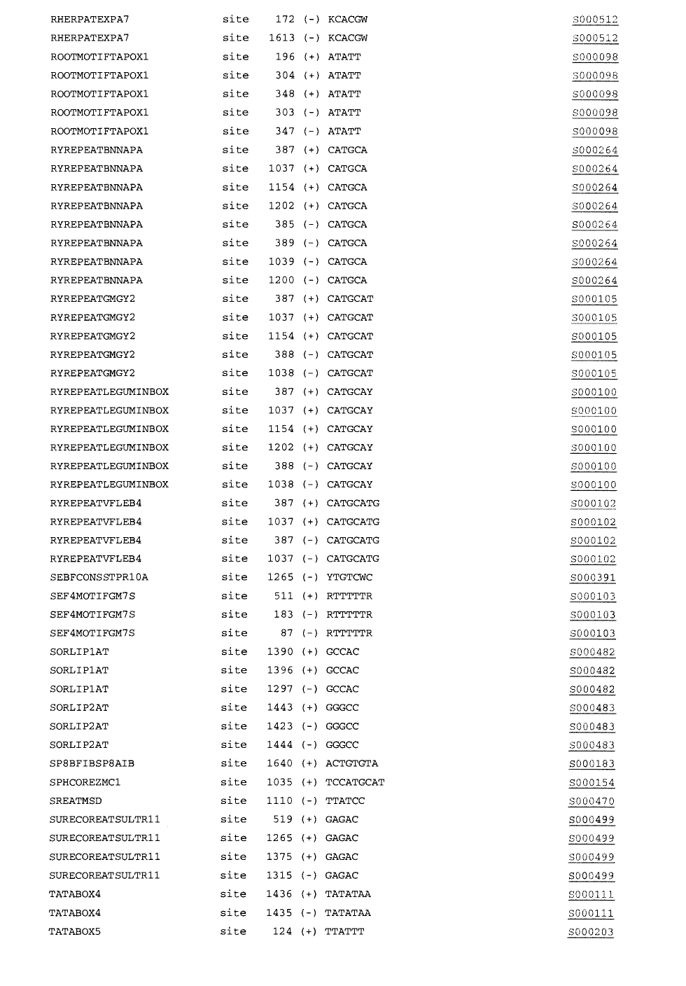

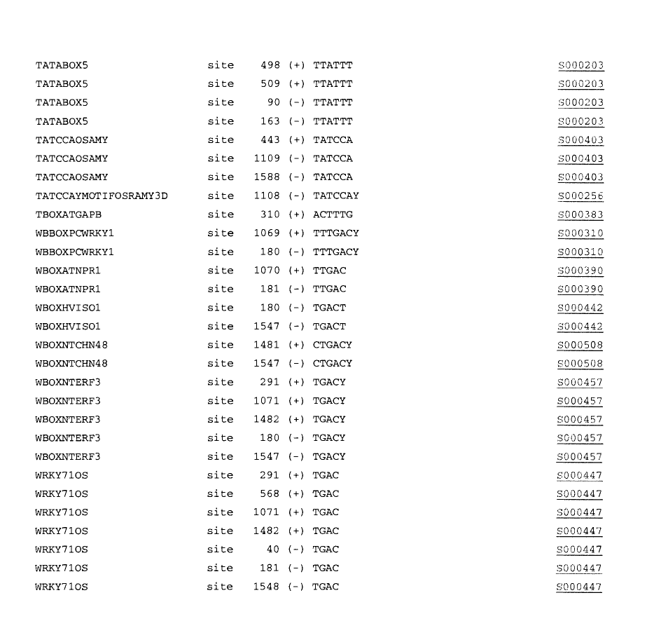

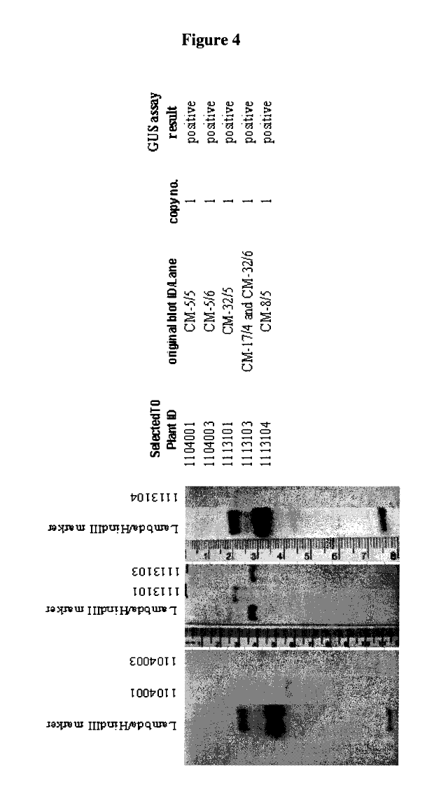

Perennial ryegrass (Lolium perenne L. cv. Tolosa) was transformed essentially as described in Bajaj et. al. (Plant Cell Reports 2006 25: 651-659). Embryogenic callus derived from mersitematic regions of the tillers of selected ryegrass lines and Agrobacterium tumefaciens strain EHA101 carrying a modified binary vector (FIG. 4) were used for transformation experiments. Embryogenic calli were immersed with overnight-grown Agrobacterium cultures for 30 minutes with continuous shaking. Calli resistant to hygromycin were selected after subculturing them on co-cultivation medium for 4 weeks. After selection, the resistant calli were subcultured on regeneration medium every 2 weeks until the plants regenerated. The regenerants that continued to grow after two or three rounds of selection proved to be stable transformants. Each regenerated plant was then multiplied on maintenance medium to produce clonal plantlets and subsequently rooted on MS medium without hormones. A rooted plant from each clone was transferred into contained glasshouse conditions while retaining a clonal counterpart in tissue culture as backup.

### Tissue Specificity of the Promoter

Tissue samples were stained in GUS staining solution (Jefferson R. A., et al., 1987. EMBO 6:3901-3907).

This ryegrass promoter has a low level of expression in all tissues of rice (leaf, root, spikelet and internodes) and throughout the rice's growth stages (Table 2; FIG. 6).

TABLE 2

| | Qualitative GUS assay | | | | |
|---|---|---|---|---|---|
| Different stages of | PRO7 | | DCaMV35S | | |
| Histochemical GUS | 1113103 | 1113104 | 1093001 | 1093004 | Wild Type |
| staining | Staining result | Staining result | Staining result | Staining result | Staining result |
| Early tillering stage | | | | | |
| Leaf | Positive | Positive | Positive | Positive | Negative |
| Active tillering stage | | | | | |
| Leaf | Positive | Positive | Positive | Positive | Negative |
| Root | Positive | Positive | Positive | Positive | Negative |
| Late tillering stage | | | | | |
| Leaf | Positive | Positive | Positive | Positive | Negative |
| Flowering stage | | | | | |
| Leaf | Positive | Positive | Positive | Positive | Negative |
| Internode | Positive | Positive | Positive | Positive | Negative |
| Root | Positive | Positive | Positive | Positive | Negative |
| Spikelet | Positive | Positive | Positive | Positive | Negative |
| Maturity stage | | | | | |
| Leaf | Positive | Positive | Positive | Positive | Negative |
| Internode | faint staining | faint staining | Positive | Positive | Negative |
| Root | no staining | Positive | Positive | Positive | Negative |
| Spikelet | Positive | Positive | Positive | Positive | Negative |

TABLE 3

| | | Activity of the extract |
|---|---|---|
| construct ID | Event ID | (pmol/min/μg) |
| PRO7 | 1113103 | 5.63 |
| PRO7 | 1113104 | 1.65 |
| D35sP | 1093001 | 0.08 |
| D35sP | 1093004 | 53.73 |
| control | Nipponbare | 0.07 |

### Deletion Analysis

Restriction enzymes can be used to make promoter deletions in order to test the control of gene expression by fragments of the promoter.

Shown below are seven examples of such fragments with the pair of restriction enzymes and the size of the resulting promoter fragment indicated.

PRO7 del SpeI-PmlI (1253 bp)
(SEQ ID NO: 10)
CTCACACCTAATTGTCCGGCCTAGATTGTCTGAAAGGGCGTCAGCTA
AGGCCATGTACAATGCAAGGTTGTTATTAGTGAGCTGCTTAAAAATA
AACCAAGTTTTTTTATTAAGCACTAGGTGGGAGGCACTGAAAATTGG
ACGGCCGGTAGTACTGACGATCGAGACTATAGATGGATCGGTCATCG
CGGTGTGTTGCTGTTTGTCTAAAACAGAGAAACCGGGATATGTAGCA
TGTTACTGCCCTGACATCTCATTTGAATTTCATGGCCGATCACACTC
ACACCTTGAATTTGCCAAGCACGTACACCTGACAGGTTTGACTACAA
CCACATATAGCAAATCTCCACGCGCGCGCACAGCTACCAAATAATTA
AGTAACGTCCAAGTGCATCGTAAAATACTGGAGTAAAATAGATGAAG
TAAATTTGGGAAATTGGTATCCGCCATAGTGGCAGGTGACTTACTTT
GCCATGAGAAAAATGCAAATCTGTCTTGTTGCTATTTAACTTGTTCA
TCAGCTATGAAGAACTATATCCTATGCTTTATTGTATGCACAGGCCG
GCACTTCGATTGGGTTATCCTAGAGCAGACGAGCGAACTCCAAAACG
AGATTGTCCATGCATCATGAGCCTGGTCCTCACCACGATTCTCTTCG
ACCAGTCGCGACCTATGCAAGGAGCCAGAGATCTCTATGGAATAATG
AACCCCAAAATCGATCGAGCAGGGGCTGATCGATCTCATGCATTCTA
AGACATGATGCTACGCGACACGATGGCGCTTAGATCTGCATGCACGG
GCGACCAAGGCCTCATTGTTAATTTCGGGCTTAAGGATGTCGGGCAC
GGTGCACGAGACAGGCTAATACTAGCGTTTTGCAAAACAGTGGCCGC
TTTTGCGCATGTCTCCTCCATCCCTTGCTGTGTGTGTTGGAGAAAAA
ACAAAGGAAAGAAACTACGCACTGAGACCTTCGAATCCGCCACTGCC
ACGACCACCGCCTCGGCCGTAGCTGGCCCTGCCCAATTATATAAGGG
CCCTCTCTGCGCATCTATTGATTTCAGCATTGCTTCTGACCAGAAGA
AGATCCCGGAAGAAACAAGATCAGAACCAGAAACCACAGCTACCGCG
AAGTGGAAGTCAGATCATACATAGAGAGCATTTTGAAGCTGTAGCTG
TTGGATAGACCGAGCTGCTCGATCAATCGTGATACACCCAAGCGATC
GGCCGAACTGTGTAGCTCGCATAGGTAGAGA
PRO7 del SpeI-StuI (564 bp)
(SEQ ID NO: 11)
CTCACACCTAATTGTCCGGCCTAGATTGTCTGAAAGGGCGTCAGCTA
AGGCCATGTACAATGCAAGGTTGTTATTAGTGAGCTGCTTAAAAATA
AACCAAGTTTTTTTATTAAGCACTAGCCTCATTGTTAATTTCGGGCT
TAAGGATGTCGGGCACGGTGCACGAGACAGGCTAATACTAGCGTTTT
GCAAAACAGTGGCCGCTTTTGCGCATCTCTCCTCCATCCCTTGCTGT
GTGTGTTGGAGAAAAAACAAAGGAAAGAAACTACGCACTGAGACCTT
CGAATCCGCCACTGCCACGACCACCGCCTCGGCCGTAGCTGGCCCTG
CCCAATTATATAAGGGCCCTCTCTGCGCATCTATTGATTTCAGCATT
GCTTCTGACCAGAAGAAGATCCCGGAAGAAACAAGATCAGAACCAGA
AACCACAGCTACCGCGAAGTGGAAGTCAGATCATACATAGAGAGCAT
TTTGAAGCTGTAGCTGTTGGATAGACCGAGCTGCTCGATCAATCGTG
ATACACCCAAGCGATCGGCCGAACTGTGTAGCTCGCATAGGTAGAGA
PRO7 del SpeI-ApaI (337 bp)
(SEQ ID NO: 12)
CTCACACCTAATTGTCCGGCCTAGATTGTCTGAAAGGGCGTCAGCTA
AGGCCATGTACAATGCAAGGTTGTTATTAGTGAGCTGCTTAAAAATA
AACCAAGTTTTTTTATTAAGCACTAGCTCTCTGCGCATCTATTGATT
TCAGCATTGCTTCTGACCAGAAGAAGATCCCGGAAGAAACAAGATCA
GAACCAGAAACCACAGCTACCGCGAAGTGGAAGTCAGATCATACATA
GAGAGCATTTTGAAGCTGTAGCTGTTGGATAGACCGAGCTGCTCGAT
CAATCGTGATACACCCAAGCGATCGGCCGAACTGTGTAGCTCGCATA
GGTAGAGA
PRO7 del PmlI-StuI (1075 bp)
(SEQ ID NO: 13)
CTCGTATGTTGTGTGGAATTGTGAGCGGATAACAATTTCACACAGGA
AACAGCTATGACCATGATTACGAATTCCCTTAATTAAGGCGCGCCCC
ATCTATCTCACACCTAATTGTCCGGCCTAGATTGTCTGAAAGGGCGT
CAGCTAAGGCCATGTACAATGCAAGGTTGTTATTAGTGAGCTGCTTA
AAAATAAACCAAGTTTTTTTATTAAGCACTAGTGCTTATTTGTATAG
GGGCTTAGTTAGGCATCTGTCTAGTGTAAATAAGGGTCGTGCTTAGT
CAAAAATTGGTTTATATTGCTAAGCATCTTTCTAAGCACTCCCCATT
GTACATATGCCCTAACTAAGTCGTTAAATTTCAATCCTCTGAAACAA
CGTACCAAAAGTTATGACCTTCTGATAATATTTACTTTGCGTGCGGT
TTTGTGAAACCTAGCTTAGTCCCAATATTTTTTAATAAATCAGAAAA
CAAAATTGAAAGATTGCATGCATGGTCTGCAGTAGCTCGAGAATGTG
GTCCACACGAAGTTGCATAATCTCATATCCAATCAAATTTGATCTAC
ATATACGCAGATATCATGGTTAGGTAGAAAATTTATTTTGGAGTTAT
TTTTGTGAGACTGTGGACACCCTCATTGTTAATTTCGGGCTTAAGGA
TGTCGGGCACGGTGCACGAGACAGGCTAATACTAGCGTTTTGCAAAA
CAGTGGCCGCTTTTGCGCATGTCTCCTCCATCCCTTGCTGTGTGTGT
TGGAGAAAAAACAAAGGAAAGAAACTACGCACTGAGACCTTCGAATC
CGCCACTGCCACGACCACCGCCTCGGCCGTAGCTGGCCCTGCCCAAT
TATATAAGGGCCCTCTCTGCGCATCTATTGATTTCAGCATTGCTTCT
GACCAGAAGAAGATCCCGGAAGAAACAAGATCAGAACCAGAAACCAC
AGCTACCGCGAAGTGGAAGTCAGATCATACATAGAGAGCATTTTGAA
GCTGTAGCTGTTGGATAGACCGAGCTGCTCGATCAATCGTGATACAC
CCAAGCGATCGGCCGAACTGTGTAGCTCGCATAGGTAGAGA
PRO7 del StuI-ApaI (1437 bp)
(SEQ ID NO: 14)
CTCACACCTAATTGTCCGGCCTAGATTGTCTGAAAGGGCGTCAGCTA
AGGCCATGTACAATGCAAGGTTGTTATTAGTGAGCTGCTTAAAAATA
AACCAAGTTTTTTTATTAAGCACTAGTGCTTATTTGTATAGGGGCTT
AGTTAGGCATCTGTCTAGTGTAAATAAGGGTCGTGCTTAGTCAAAAA
TTGGTTTATATTGCTAAGCATCTTTCTAAGCACTCCCCATTGTACAT
ATGCCCTAACTAAGTCGTTAAATTTCAATCCTCTGAAACAACGTACC
AAAAGTTATGACCTTCTGATAATATTTACTTTGCGTGCGGTTTTGTG
AAACCTAGCTTAGTCCCAATATTTTTTAATAAATCAGAAAACAAAAT
TGAAAGATTGCATGCATGGTCTGCAGTAGCTCGAGAATGTGGTCCAC
ACGAAGTTGCATAATCTCATATCCAATCAAATTTGATCTACATATAC
GCAGATATCATGGTTAGGTAGAAAATTTATTTTGGAGTTATTTTTGT
GAGACTGTGGACACGTGGGAGGCACTGAAAATTGGACGGCCGGTAGT
ACTGACGATCGAGACTATAGATGGATCGGTCATCGCGGTGTGTTGCT
GTTTGTCTAAAACAGAGAAACCGGGATATGTAGCATGTTACTGCCCT
GACATCTCATTTGAATTTCATGGCCGATCACACTCACACCTTGAATT
TGCCAAGCACGTACACCTGACAGGTTTGACTACAACCACATATAGCA
AATCTCCACGCGCGCGCACAGCTACCAAATAATTAAGTAACGTCCAA
GTGCATCGTAAAATACTGGAGTAAAATAGATGAAGTAAATTTGGGAA
ATTGGTATCCGCCATAGTGGCAGGTGACTTACTTTGCCATGAGAAAA
ATGCAAATCTGTCTTGTTGCTATTTAACTTGTTCATCAGCTATGAAG
AACTATATCCTATGCTTTATTGTATGCACAGGCCGGCACTTCGATTG
GGTTATCCTAGAGCAGACGAGCGAACTCCAAAACGAGATTGTCCATG
CATGATGAGCCTGGTCCTCACCACGATTCTCTTCGACCAGTCGCGAC
CTATGCAAGGAGCCAGAGATCTCTATGGAATAATGAACCCCAAAATC
GATCGAGCAGGGGCTGATCGATCTCATGCATTCTAAGACATGATGCT
ACGCGACACGATGGCGCTTAGATCTGCATGCACGGGCGACCAAGGCT
CTCTGCGCATCTATTGATTTCAGCATTGCTTCTGACCAGAAGAAGAT
CCCGGAAGAAACAAGATCAGAACCAGAAACCACAGCTACCGCGAAGT
GGAAGTCAGATCATACATAGAGAGCATTTTGAAGCTGTAGCTGTTGG
ATAGACCGAGCTGCTCGATCAATCGTGATACACCCAAGCGATCGGCC
GAACTGTGTAGCTCGCATAGGTAGAGA
PRO7 del PmlI-ApaI (748bp)
(SEQ ID NO: 15)
CTCACACCTAATTGTCCGGCCTAGATTGTCTGAAAGGGCGTCAGCTA
AGGCCATGTACAATGCAAGGTTGTTATTAGTGAGCTGCTTAAAAATA
AACCAAGTTTTTTTATTAAGCACTAGTGCTTATTTGTATAGGGGCTT
AGTTAGGCATCTGTCTAGTGTAAATAAGGGTCGTGCTTAGTCAAAAA
TTGGTTTATATTGCTAAGCATCTTTCTAAGCACTCCCCATTGTACAT
ATGCCCTAACTAAGTCGTTAAATTTCAATCCTCTGAAACAACGTACC
AAAAGTTATGACCTTCTGATAATATTTACTTTGCGTGCGGTTTTGTG
AAACCTAGCTTAGTCCCAATATTTTTTAATAAATCAGAAAACAAAAT
TGAAAGATTGCATGCATGGTCTGCAGTAGCTCGAGAATGTGGTCCAC
ACGAAGTTGCATAATCTCATATCCAATCAAATTTGATCTACATATAC
GCAGATATCATGGTTAGGTAGAAAATTTATTTTGGAGTTATTTTTGT
GAGACTGTGGACACCTCTCTGCGCATCTATTGATTTCAGCATTGCTT
CTGACCAGAAGAAGATCCCGGAAGAAACAAGATCAGAACCAGAAACC
ACAGCTACCGCGAAGTGGAAGTCAGATCATACATAGAGAGCATTTTG
AAGCTGTAGCTGTTGGATAGACCGAGCTGCTCGATCAATCGTGATAC
ACCCAAGCGATCGGCCGAACTGTGTAGCTCGCATAGGTAGAGA
PRO7 del PstI (1264 bp)
(SEQ ID NO: 16)
TTCTCTACCTATGCGAGCTACACAGTTCGGCCGATCGCTTGGGTGTA
TCACGATTGATCGAGCAGCTCGGTCTATCCAACAGCTACAGCTTCAA
AATGCTCTCTATGTATGATCTGACTTCCACTTCGCGGTAGCTGTGGT
TTCTGGTTCTGATCTTGTTTCTTCCGGGATCTTCTTCTGGTCAGAAG
CAATGCTGAAATCAATAGATGCGCAGAGAGGGCCCTTATATAATTGG
GCAGGGCCAGCTACGGCCGAGGCGGTGGTCGTGGCAGTGGCGGATTC
GAAGGTCTCAGTGCGTAGTTTCTTTCCTTTGTTTTTTCTCCAACACA
CACAGCAAGGGATGGAGGAGACATGCGCAAAAGCGGCCACTGTTTTG
CAAAACGCTAGTATTAGCCTGTCTCGTGCACCGTGCCCGACATCCTT
AAGCCCGAAATTAACAATGAGGCCTTGGTCGCCCGTGCATGCAGATC
TAAGCGCCATCGTGTCGCGTAGCATCATGTCTTAGAATGCATGAGAT
CGATCAGCCCCTGCTCGATCGATTTTGGGGTTCATTATTCCATAGAG
ATCTCTGGCTCCTTGCATAGGTCGCGACTGGTCGAAGAGAATCGTGG
TGAGGACCAGGCTCATCATGCATGGACAATCTCGTTTTGGAGTTCGC
TCGTCTGCTCTAGGATAACCCAATCGAAGTGCCGGCCTGTGCATACA
ATAAAGCATAGGATATAGTTCTTCATAGCTGATGAACAAGTTAAATA
GCAACAAGACAGATTTGCATTTTTCTCATGGCAAAGTAAGTCACCTG
CCACTATGGCGGATACCAATTTCCCAAATTTACTTCATCTATTTTAC
TCCAGTATTTTACGATGCACTTGGACGTTACTTAATTATTTGGTAGC
TGTGCGCGCGCGTGGAGATTTGCTATATGTGGTTGTAGTCAAACCTG
TCAGGTGTACGTGCTTGGCAAATTCAAGGTGTGAGTGTGATCGGCCA
TGAAATTCAAATGAGATGTCAGGGCAGTAACATGCTACATATCCCGG
TTTCTCTGTTTTAGACAAACAGCAACACACCGCGATGACCGATCCAT
CTATAGTCTCGATCGTCAGTACTACCGGCCGTCCAATTTTCAGTGCC
TCCCACGTGTCCACAGTCTCACAAAAATAACTCCAAAATAAATTTTC
TACCTAACCATGATATCTGCGTATATGTAGATCAAATTTGATTGGAT
ATGAGATTATGCAACTTCGTGTGGACCACATTCTCGAGCTAC

Such fragments may be tested by fusion of the fragments to reporter genes such as the GUS reporter gene and histochemical staining of transformed tissue by standard methods such as those described above.

### Preparation of a Promoter-Deletion Reporter Construct

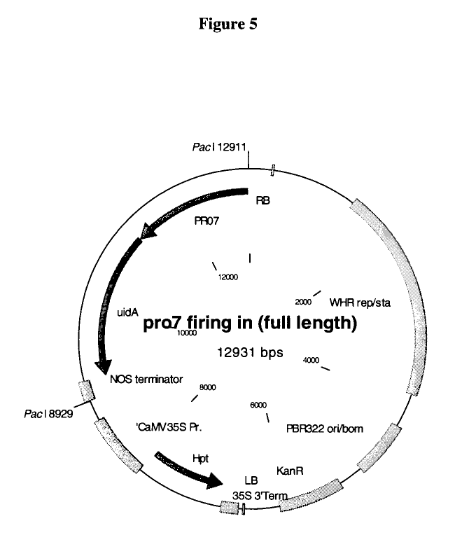

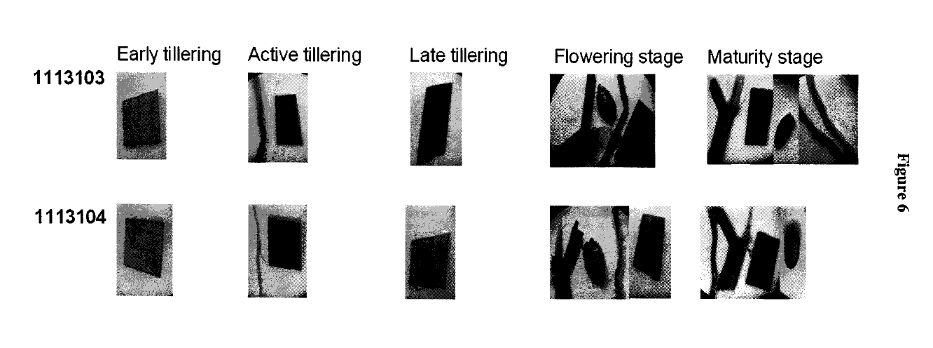

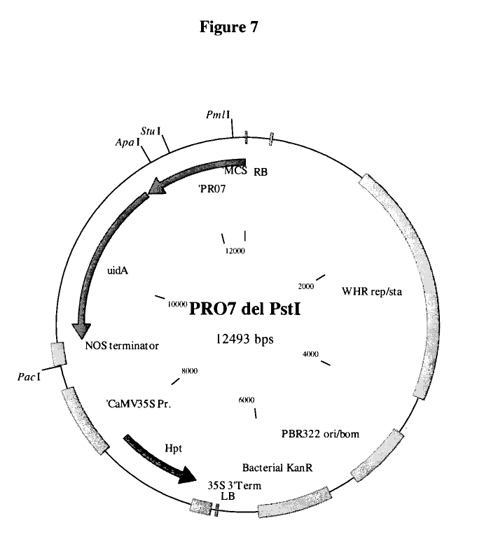

The PRO7-AG binary construct (shown in FIG. 5; SEQ ID NO:2) was digested with the restriction enzyme PstI and then electrophoresed on an agarose gel to separate the deleted promoter fragment from the vector. The vector was extracted from the gel using Qiagen Gel-extraction kit and re-ligated to result in PRO7ΔPstI-AK (FIG. 7). The sequence of the construct is shown in SEQ ID NO: 7.

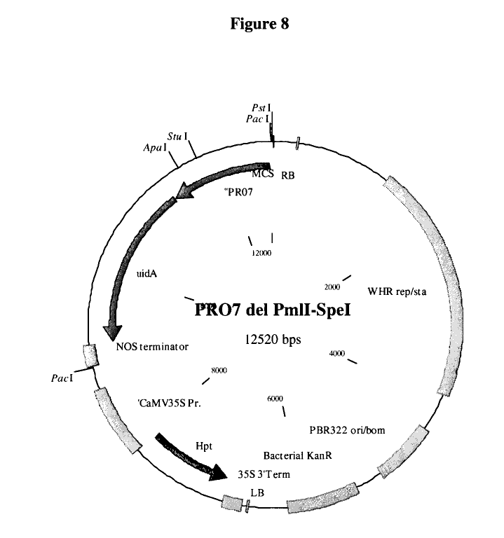

PRO7ΔPmlI-SpeI-AL was generated by digesting PRO7-AG binary construct with the restriction enzymes PmlI and SpeI, which was then blunt ended using Klenow Fragment. The blunt-ended digested products were electrophoresed on an agarose gel to separate the deleted promoter fragment from the vector. The vector was extracted from the gel using Qiagen Gel-extraction kit and re-ligated to result in PRO7ΔPmlI-AL (FIG. 8). The sequence of the construct is shown in SEQ ID NO: 8.

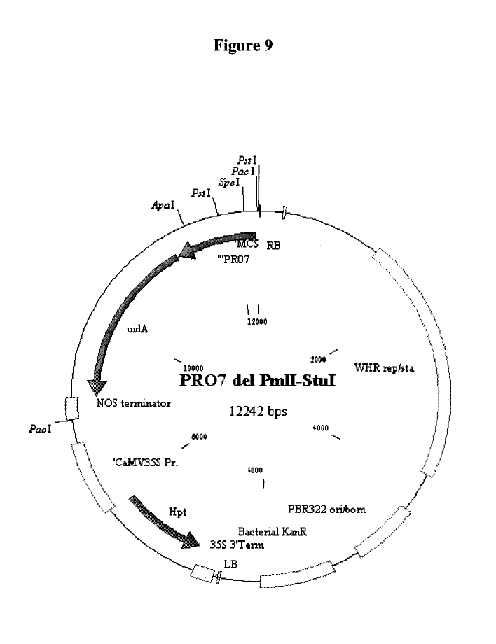

Similarly, PRO7ΔPmlI-StuI-AM was generated by digesting PRO7-AG binary construct with the restriction enzymes PmlI and StuI and then electrophoresed on an agarose gel to separate the deleted promoter fragment from the vector. The vector was extracted from the gel using Qiagen Gel-extraction kit and re-ligated to result in PRO7ΔPmlI-StuI-AM (FIG. 9). The sequence of the construct is shown in SEQ ID NO: 9.

qRT-pCR Analysis

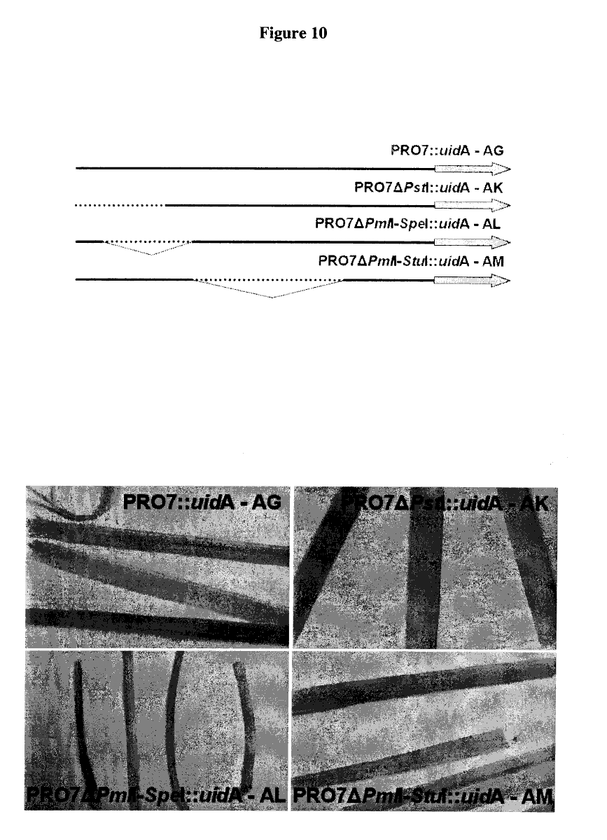

Plant leaves were frozen in liquid nitrogen and then ground to a fine powder using a mortar and pestle and liquid nitrogen. Total RNA was extracted from the ground leaves using Qiagen Plant RNeasy kit and RNAseI-free DNAse I (Qiagen). First strand cDNA was synthesised from 5 μg total RNA using SuperscriptIII reverse transcriptase (Invitrogen) using manufacturer's protocol. A 15 μL aliquot of the first-strand cDNA was diluted with 35 μL sterile water and then used for quantitative real-time polymerise chain (qRT-PCR) analysis. Concentration of the uidA transcript was measured for each construct in ABI Prism 7700 (Applied BioSystems) using the Sybr Green technology in 25 μL PCR-mix using 5 μL of the diluted first-strand cDNA. The quantities were normalised against the level of chlorophyll AB binding protein gene transcript in the same sample and relative gene expression levels determined (see FIG. 10).

The results in FIG. 10 show that expression of GUS activity is reduced with each deletion.

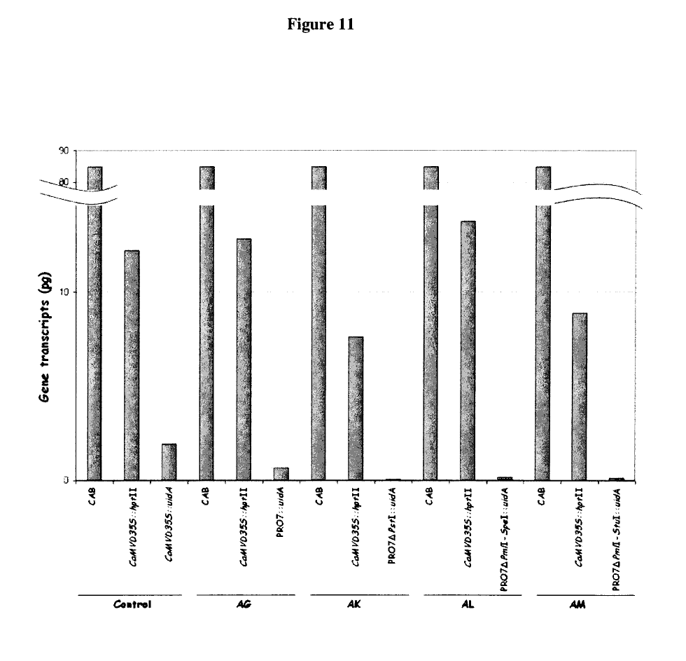

Similarly expression of uidA is reduced with each deletion as shown in FIG. 11.

The results in FIGS. 10 and 11 also show that the promoter of the invention, and fragments thereof, is capable of controlling transcription of an operably linked polynucleotide in ryegrass.

The above Examples illustrate practice of the invention. It will be appreciated by those skilled in the art that numerous variations and modifications may be made without departing from the spirit and scope of the invention.

1-18. (canceled)

19. An isolated promoter polynucleotide comprising at least one of:
a) the sequence of SEQ ID NO:1;
b) a sequence with at least 70% identity to the sequence of SEQ ID NO:1;
c) a sequence fragment consisting of at least 50 contiguous nucleotides of the sequence of SEQ ID NO:1;
d) a sequence fragment consisting of at least 50 contiguous nucleotides of the sequence
of b);
e) a sequence with at least 70% identity to the sequence of c); and
f) a sequence that is a complement of any one of a) to e)
wherein the promoter polynucleotide is capable of controlling transcription of an operably linked polynucleotide in a plant.

20. The isolated promoter polynucleotide of claim 19, comprising a sequence with at least 70% identity to the sequence of SEQ ID NO:1.

21. The isolated promoter polynucleotide of claim 19, comprising a sequence fragment consisting of at least 50 contiguous nucleotides of the sequence of
SEQ ID NO:1.

22. The isolated promoter polynucleotide of claim 19, comprising a sequence fragment consisting of a sequence selected from any one of SEQ ID NO:10 to SEQ ID NO:16.

23. The isolated promoter polynucleotide of claim 19, comprising the sequence of SEQ ID NO:1.

24. The isolated promoter polynucleotide of claim 19, consisting of the sequence of SEQ ID NO:1.

25. The isolated promoter polynucleotide of claim 19, which is capable of controlling transcription of an operably linked polynucleotide sequence in a plant in at least one of leaves, internodes, roots and flowers.

26. The isolated promoter polynucleotide of claim 19, which is capable of controlling transcription of an operably linked polynucleotide sequence constitutively in substantially all tissues of a plant.

27. A genetic construct comprising a promoter polynucleotide of claim 19.

28. The genetic construct comprising a promoter polynucleotide of claim 19, in which the promoter polynucleotide is operably linked to a polynucleotide sequence to be expressed.

29. A plant cell or plant transformed with the promoter polynucleotide of claim 19.

30. A plant cell or plant transformed with a genetic construct comprising a promoter polynucleotide of claim 19.

31. A method for producing a plant cell or plant with modified expression of at least one polynucleotide, the method comprising:
(a) transforming plant cell or plant with a promoter polynucleotide of claim 19, and
(b) the cultivating the transgenic plant cell or plant under conditions conducive for the promoter polynucleotide to drive transcription.

32. A method for producing a plant cell or plant with modified expression of at least one polynucleotide, the method comprising:
(a) transforming plant cell or plant with a genetic construct comprising a promoter polynucleotide of claim 19, and
(b) the cultivating the transgenic plant cell or plant under conditions conducive for the promoter polynucleotide to drive transcription.

33. A method for producing a plant cell or plant with a modified phenotype, the method including the stable incorporation into the genome of the plant, a promoter polynucleotide of claim 19.

34. A method for producing a plant cell or plant with a modified phenotype, the method including the stable incorporation into the genome of the plant, a genetic construct comprising a promoter polynucleotide of claim 19.

35. A seed, propagule, progeny or part of a plant transformed with the promoter polynucleotide of claim 19, wherein the seed, propagule, progeny or part is transformed with the polynucleotide.

36. A seed, propagule, progeny or part of a plant transformed with a genetic construct comprising a promoter polynucleotide of claim 19, wherein the seed, propagule, progeny or part is transformed with the genetic construct.


SEQUENCE LISTING (16 sequences)

SEQ ID NO: 1 - DNA, 1664 bp, Lolium perenne
ctcacacctaattgtccggcctagattgtctgaaagggcgtcagctaaggccatgtacaatgcaaggttgttattagtgagctgcttaaaaataaaccaagtttttttattaagcactagtgcttatttgtataggggcttagttaggcatctgtctagtgtaaataagggtcgtgcttagtcaaaaattggtttatattgctaagcatctttctaagcactccccattgtacatatgccctaactaagtcgttaaatttcaatcctctgaaacaacgtaccaaaagttatgaccttctgataatatttactttgcgtgcggttttgtgaaacctagcttagtcccaatattttttaataaatcagaaaacaaaattgaaagattgcatgcatggtctgcagtagctcgagaatgtggtccacacgaagttgcataatctcatatccaatcaaatttgatctacatatacgcagatatcatggttaggtagaaaatttattttggagttatttttgtgagactgtggacacgtgggaggcactgaaaattggacggccggtagtactgacgatcgagactatagatggatcggtcatcgcggtgtgttgctgtttgtctaaaacagagaaaccgggatatgtagcatgttactgccctgacatctcatttgaatttcatggccgatcacactcacaccttgaatttgccaagcacgtacacctgacaggtttgactacaaccacatatagcaaatctccacgcgcgcgcacagctaccaaataattaagtaacgtccaagtgcatcgtaaaatactggagtaaaatagatgaagtaaatttgggaaattggtatccgccatagtggcaggtgacttactttgccatgagaaaaatgcaaatctgtcttgttgctatttaacttgttcatcagctatgaagaactatatcctatgctttattgtatgcacaggccggcacttcgattgggttatcctagagcagacgagcgaactccaaaacgagattgtccatgcatgatgagcctggtcctcaccacgattctcttcgaccagtcgcgacctatgcaaggagccagagatctctatggaataatgaaccccaaaatcgatcgagcaggggctgatcgatctcatgcattctaagacatgatgctacgcgacacgatggcgcttagatctgcatgcacgggcgaccaaggcctcattgttaatttcgggcttaaggatgtcgggcacggtgcacgagacaggctaatactagcgttttgcaaaacagtggccgcttttgcgcatgtctcctccatcccttgctgtgtgtgttggagaaaaaacaaaggaaagaaactacgcactgagaccttcgaatccgccactgccacgaccaccgcctcggccgtagctggccctgcccaattatataagggccctctctgcgcatctattgatttcagcattgcttctgaccagaagaagatcccggaagaaacaagatcagaaccagaaaccacagctaccgcgaagtggaagtcagatcatacatagagagcattttgaagctgtagctgttggatagaccgagctgctcgatcaatcgtgatacacccaagcgatcggccgaactgtgtagctcgcataggtagaga

SEQ ID NO: 2 - DNA, 12931 bp, Artificial
  Vector
ggaattcgatatcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccccccggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcccttaattaagatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtggtaccggttcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggacgagtcgacggttctgtaactatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttaccctgtagggatccatggtccggaccataagttctctacctatgcgagctacacagttcggccgatcgcttgggtgtatcacgattgatcgagcagctcggtctatccaacagctacagcttcaaaatgctctctatgtatgatctgacttccacttcgcggtagctgtggtttctggttctgatcttgtttcttccgggatcttcttctggtcagaagcaatgctgaaatcaatagatgcgcagagagggcccttatataattgggcagggccagctacggccgaggcggtggtcgtggcagtggcggattcgaaggtctcagtgcgtagtttctttcctttgttttttctccaacacacacagcaagggatggaggagacatgcgcaaaagcggccactgttttgcaaaacgctagtattagcctgtctcgtgcaccgtgcccgacatccttaagcccgaaattaacaatgaggccttggtcgcccgtgcatgcagatctaagcgccatcgtgtcgcgtagcatcatgtcttagaatgcatgagatcgatcagcccctgctcgatcgattttggggttcattattccatagagatctctggctccttgcataggtcgcgactggtcgaagagaatcgtggtgaggaccaggctcatcatgcatggacaatctcgttttggagttcgctcgtctgctctaggataacccaatcgaagtgccggcctgtgcatacaataaagcataggatatagttcttcatagctgatgaacaagttaaatagcaacaagacagatttgcatttttctcatggcaaagtaagtcacctgccactatggcggataccaatttcccaaatttacttcatctattttactccagtattttacgatgcacttggacgttacttaattatttggtagctgtgcgcgcgcgtggagatttgctatatgtggttgtagtcaaacctgtcaggtgtacgtgcttggcaaattcaaggtgtgagtgtgatcggccatgaaattcaaatgagatgtcagggcagtaacatgctacatatcccggtttctctgttttagacaaacagcaacacaccgcgatgaccgatccatctatagtctcgatcgtcagtactaccggccgtccaattttcagtgcctcccacgtgtccacagtctcacaaaaataactccaaaataaattttctacctaaccatgatatctgcgtatatgtagatcaaatttgattggatatgagattatgcaacttcgtgtggaccacattctcgagctactgcagaccatgcatgcaatctttcaattttgttttctgatttattaaaaaatattgggactaagctaggtttcacaaaaccgcacgcaaagtaaatattatcagaaggtcataacttttggtacgttgtttcagaggattgaaatttaacgacttagttagggcatatgtacaatggggagtgcttagaaagatgcttagcaatataaaccaatttttgactaagcacgacccttatttacactagacagatgcctaactaagcccctatacaaataagcactagtgcttaataaaaaaacttggtttatttttaagcagctcactaataacaaccttgcattgtacatggccttagctgacgccctttcagacaatctaggccggacaattaggtgtgagatagatggggcgcgccttaattaaggcgcgccctgca

SEQ ID NO: 3 - DNA, 18 bp, Artificial
  Primer
ctaggtccagagtgtagg

SEQ ID NO: 4 - DNA, 20 bp, Artificial
  Primer
ttccaccgcccgcacttgac

SEQ ID NO: 5 - DNA, 19 bp, Artificial
  Primer
ctcacacctaattgtccgg

SEQ ID NO: 6 - DNA, 20 bp, Artificial
  Primer
tctctacctatgcgagctac

SEQ ID NO: 7 - DNA, 12493 bp, Artificial
  Vector
ggaattcgatatcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccccccggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcccttaattaagatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtggtaccggttcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggacgagtcgacggttctgtaactatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttaccctgtagggatccatggtccggaccataagttctctacctatgcgagctacacagttcggccgatcgcttgggtgtatcacgattgatcgagcagctcggtctatccaacagctacagcttcaaaatgctctctatgtatgatctgacttccacttcgcggtagctgtggtttctggttctgatcttgtttcttccgggatcttcttctggtcagaagcaatgctgaaatcaatagatgcgcagagagggcccttatataattgggcagggccagctacggccgaggcggtggtcgtggcagtggcggattcgaaggtctcagtgcgtagtttctttcctttgttttttctccaacacacacagcaagggatggaggagacatgcgcaaaagcggccactgttttgcaaaacgctagtattagcctgtctcgtgcaccgtgcccgacatccttaagcccgaaattaacaatgaggccttggtcgcccgtgcatgcagatctaagcgccatcgtgtcgcgtagcatcatgtcttagaatgcatgagatcgatcagcccctgctcgatcgattttggggttcattattccatagagatctctggctccttgcataggtcgcgactggtcgaagagaatcgtggtgaggaccaggctcatcatgcatggacaatctcgttttggagttcgctcgtctgctctaggataacccaatcgaagtgccggcctgtgcatacaataaagcataggatatagttcttcatagctgatgaacaagttaaatagcaacaagacagatttgcatttttctcatggcaaagtaagtcacctgccactatggcggataccaatttcccaaatttacttcatctattttactccagtattttacgatgcacttggacgttacttaattatttggtagctgtgcgcgcgcgtggagatttgctatatgtggttgtagtcaaacctgtcaggtgtacgtgcttggcaaattcaaggtgtgagtgtgatcggccatgaaattcaaatgagatgtcagggcagtaacatgctacatatcccggtttctctgttttagacaaacagcaacacaccgcgatgaccgatccatctatagtctcgatcgtcagtactaccggccgtccaattttcagtgcctcccacgtgtccacagtctcacaaaaataactccaaaataaattttctacctaaccatgatatctgcgtatatgtagatcaaatttgattggatatgagattatgcaacttcgtgtggaccacattctcgagctac

SEQ ID NO: 8 - DNA, 12520 bp, Artificial
  Vector
ggaattcgatatcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccccccggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcccttaattaagatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtggtaccggttcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggacgagtcgacggttctgtaactatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttaccctgtagggatccatggtccggaccataagttctctacctatgcgagctacacagttcggccgatcgcttgggtgtatcacgattgatcgagcagctcggtctatccaacagctacagcttcaaaatgctctctatgtatgatctgacttccacttcgcggtagctgtggtttctggttctgatcttgtttcttccgggatcttcttctggtcagaagcaatgctgaaatcaatagatgcgcagagagggcccttatataattgggcagggccagctacggccgaggcggtggtcgtggcagtggcggattcgaaggtctcagtgcgtagtttctttcctttgttttttctccaacacacacagcaagggatggaggagacatgcgcaaaagcggccactgttttgcaaaacgctagtattagcctgtctcgtgcaccgtgcccgacatccttaagcccgaaattaacaatgaggccttggtcgcccgtgcatgcagatctaagcgccatcgtgtcgcgtagcatcatgtcttagaatgcatgagatcgatcagcccctgctcgatcgattttggggttcattattccatagagatctctggctccttgcataggtcgcgactggtcgaagagaatcgtggtgaggaccaggctcatcatgcatggacaatctcgttttggagttcgctcgtctgctctaggataacccaatcgaagtgccggcctgtgcatacaataaagcataggatatagttcttcatagctgatgaacaagttaaatagcaacaagacagatttgcatttttctcatggcaaagtaagtcacctgccactatggcggataccaatttcccaaatttacttcatctattttactccagtattttacgatgcacttggacgttacttaattatttggtagctgtgcgcgcgcgtggagatttgctatatgtggttgtagtcaaacctgtcaggtgtacgtgcttggcaaattcaaggtgtgagtgtgatcggccatgaaattcaaatgagatgtcagggcagtaacatgctacatatcccggtttctctgttttagacaaacagcaacacaccgcgatgaccgatccatctatagtctcgatcgtcagtactaccggccgtccaattttcagtgcctcccacctagtgcttaataaaaaaacttggtttatttttaagcagctcactaataacaaccttgcattgtacatggccttagctgacgccctttcagacaatctaggccggacaattaggtgtgagatagatggggcgcgccttaattaaggcgcgccctgca

SEQ ID NO: 9 - DNA, 12242 bp, Artificial
  Vector
ggaattcgatatcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccccccggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcccttaattaagatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtggtaccggttcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggacgagtcgacggttctgtaactatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttaccctgtagggatccatggtccggaccataagttctctacctatgcgagctacacagttcggccgatcgcttgggtgtatcacgattgatcgagcagctcggtctatccaacagctacagcttcaaaatgctctctatgtatgatctgacttccacttcgcggtagctgtggtttctggttctgatcttgtttcttccgggatcttcttctggtcagaagcaatgctgaaatcaatagatgcgcagagagggcccttatataattgggcagggccagctacggccgaggcggtggtcgtggcagtggcggattcgaaggtctcagtgcgtagtttctttcctttgttttttctccaacacacacagcaagggatggaggagacatgcgcaaaagcggccactgttttgcaaaacgctagtattagcctgtctcgtgcaccgtgcccgacatccttaagcccgaaattaacaatgagggtgtccacagtctcacaaaaataactccaaaataaattttctacctaaccatgatatctgcgtatatgtagatcaaatttgattggatatgagattatgcaacttcgtgtggaccacattctcgagctactgcagaccatgcatgcaatctttcaattttgttttctgatttattaaaaaatattgggactaagctaggtttcacaaaaccgcacgcaaagtaaatattatcagaaggtcataacttttggtacgttgtttcagaggattgaaatttaacgacttagttagggcatatgtacaatggggagtgcttagaaagatgcttagcaatataaaccaatttttgactaagcacgacccttatttacactagacagatgcctaactaagcccctatacaaataagcactagtgcttaataaaaaaacttggtttatttttaagcagctcactaataacaaccttgcattgtacatggccttagctgacgccctttcagacaatctaggccggacaattaggtgtgagatagatggggcgcgccttaattaaggcgcgccctgca

SEQ ID NO: 10 - DNA, 1253 bp, Lolium perenne
ctcacacctaattgtccggcctagattgtctgaaagggcgtcagctaaggccatgtacaatgcaaggttgttattagtgagctgcttaaaaataaaccaagtttttttattaagcactaggtgggaggcactgaaaattggacggccggtagtactgacgatcgagactatagatggatcggtcatcgcggtgtgttgctgtttgtctaaaacagagaaaccgggatatgtagcatgttactgccctgacatctcatttgaatttcatggccgatcacactcacaccttgaatttgccaagcacgtacacctgacaggtttgactacaaccacatatagcaaatctccacgcgcgcgcacagctaccaaataattaagtaacgtccaagtgcatcgtaaaatactggagtaaaatagatgaagtaaatttgggaaattggtatccgccatagtggcaggtgacttactttgccatgagaaaaatgcaaatctgtcttgttgctatttaacttgttcatcagctatgaagaactatatcctatgctttattgtatgcacaggccggcacttcgattgggttatcctagagcagacgagcgaactccaaaacgagattgtccatgcatgatgagcctggtcctcaccacgattctcttcgaccagtcgcgacctatgcaaggagccagagatctctatggaataatgaaccccaaaatcgatcgagcaggggctgatcgatctcatgcattctaagacatgatgctacgcgacacgatggcgcttagatctgcatgcacgggcgaccaaggcctcattgttaatttcgggcttaaggatgtcgggcacggtgcacgagacaggctaatactagcgttttgcaaaacagtggccgcttttgcgcatgtctcctccatcccttgctgtgtgtgttggagaaaaaacaaaggaaagaaactacgcactgagaccttcgaatccgccactgccacgaccaccgcctcggccgtagctggccctgcccaattatataagggccctctctgcgcatctattgatttcagcattgcttctgaccagaagaagatcccggaagaaacaagatcagaaccagaaaccacagctaccgcgaagtggaagtcagatcatacatagagagcattttgaagctgtagctgttggatagaccgagctgctcgatcaatcgtgatacacccaagcgatcggccgaactgtgtagctcgcataggtagaga

SEQ ID NO: 11 - DNA, 564 bp, Lolium perenne
ctcacacctaattgtccggcctagattgtctgaaagggcgtcagctaaggccatgtacaatgcaaggttgttattagtgagctgcttaaaaataaaccaagtttttttattaagcactagcctcattgttaatttcgggcttaaggatgtcgggcacggtgcacgagacaggctaatactagcgttttgcaaaacagtggccgcttttgcgcatgtctcctccatcccttgctgtgtgtgttggagaaaaaacaaaggaaagaaactacgcactgagaccttcgaatccgccactgccacgaccaccgcctcggccgtagctggccctgcccaattatataagggccctctctgcgcatctattgatttcagcattgcttctgaccagaagaagatcccggaagaaacaagatcagaaccagaaaccacagctaccgcgaagtggaagtcagatcatacatagagagcattttgaagctgtagctgttggatagaccgagctgctcgatcaatcgtgatacacccaagcgatcggccgaactgtgtagctcgcataggtagaga

SEQ ID NO: 12 - DNA, 337 bp, Lolium perenne
ctcacacctaattgtccggcctagattgtctgaaagggcgtcagctaaggccatgtacaatgcaaggttgttattagtgagctgcttaaaaataaaccaagtttttttattaagcactagctctctgcgcatctattgatttcagcattgcttctgaccagaagaagatcccggaagaaacaagatcagaaccagaaaccacagctaccgcgaagtggaagtcagatcatacatagagagcattttgaagctgtagctgttggatagaccgagctgctcgatcaatcgtgatacacccaagcgatcggccgaactgtgtagctcgcataggtagaga

SEQ ID NO: 13 - DNA, 1075 bp, Lolium perenne
ctcgtatgttgtgtggaattgtgagcggataacaatttcacacaggaaacagctatgaccatgattacgaattcccttaattaaggcgcgccccatctatctcacacctaattgtccggcctagattgtctgaaagggcgtcagctaaggccatgtacaatgcaaggttgttattagtgagctgcttaaaaataaaccaagtttttttattaagcactagtgcttatttgtataggggcttagttaggcatctgtctagtgtaaataagggtcgtgcttagtcaaaaattggtttatattgctaagcatctttctaagcactccccattgtacatatgccctaactaagtcgttaaatttcaatcctctgaaacaacgtaccaaaagttatgaccttctgataatatttactttgcgtgcggttttgtgaaacctagcttagtcccaatattttttaataaatcagaaaacaaaattgaaagattgcatgcatggtctgcagtagctcgagaatgtggtccacacgaagttgcataatctcatatccaatcaaatttgatctacatatacgcagatatcatggttaggtagaaaatttattttggagttatttttgtgagactgtggacaccctcattgttaatttcgggcttaaggatgtcgggcacggtgcacgagacaggctaatactagcgttttgcaaaacagtggccgcttttgcgcatgtctcctccatcccttgctgtgtgtgttggagaaaaaacaaaggaaagaaactacgcactgagaccttcgaatccgccactgccacgaccaccgcctcggccgtagctggccctgcccaattatataagggccctctctgcgcatctattgatttcagcattgcttctgaccagaagaagatcccggaagaaacaagatcagaaccagaaaccacagctaccgcgaagtggaagtcagatcatacatagagagcattttgaagctgtagctgttggatagaccgagctgctcgatcaatcgtgatacacccaagcgatcggccgaactgtgtagctcgcataggtagaga

SEQ ID NO: 14 - DNA, 1437 bp, Lolium perenne
ctcacacctaattgtccggcctagattgtctgaaagggcgtcagctaaggccatgtacaatgcaaggttgttattagtgagctgcttaaaaataaaccaagtttttttattaagcactagtgcttatttgtataggggcttagttaggcatctgtctagtgtaaataagggtcgtgcttagtcaaaaattggtttatattgctaagcatctttctaagcactccccattgtacatatgccctaactaagtcgttaaatttcaatcctctgaaacaacgtaccaaaagttatgaccttctgataatatttactttgcgtgcggttttgtgaaacctagcttagtcccaatattttttaataaatcagaaaacaaaattgaaagattgcatgcatggtctgcagtagctcgagaatgtggtccacacgaagttgcataatctcatatccaatcaaatttgatctacatatacgcagatatcatggttaggtagaaaatttattttggagttatttttgtgagactgtggacacgtgggaggcactgaaaattggacggccggtagtactgacgatcgagactatagatggatcggtcatcgcggtgtgttgctgtttgtctaaaacagagaaaccgggatatgtagcatgttactgccctgacatctcatttgaatttcatggccgatcacactcacaccttgaatttgccaagcacgtacacctgacaggtttgactacaaccacatatagcaaatctccacgcgcgcgcacagctaccaaataattaagtaacgtccaagtgcatcgtaaaatactggagtaaaatagatgaagtaaatttgggaaattggtatccgccatagtggcaggtgacttactttgccatgagaaaaatgcaaatctgtcttgttgctatttaacttgttcatcagctatgaagaactatatcctatgctttattgtatgcacaggccggcacttcgattgggttatcctagagcagacgagcgaactccaaaacgagattgtccatgcatgatgagcctggtcctcaccacgattctcttcgaccagtcgcgacctatgcaaggagccagagatctctatggaataatgaaccccaaaatcgatcgagcaggggctgatcgatctcatgcattctaagacatgatgctacgcgacacgatggcgcttagatctgcatgcacgggcgaccaaggctctctgcgcatctattgatttcagcattgcttctgaccagaagaagatcccggaagaaacaagatcagaaccagaaaccacagctaccgcgaagtggaagtcagatcatacatagagagcattttgaagctgtagctgttggatagaccgagctgctcgatcaatcgtgatacacccaagcgatcggccgaactgtgtagctcgcataggtagaga

SEQ ID NO: 15 - DNA, 748 bp, Lolium perenne
ctcacacctaattgtccggcctagattgtctgaaagggcgtcagctaaggccatgtacaatgcaaggttgttattagtgagctgcttaaaaataaaccaagtttttttattaagcactagtgcttatttgtataggggcttagttaggcatctgtctagtgtaaataagggtcgtgcttagtcaaaaattggtttatattgctaagcatctttctaagcactccccattgtacatatgccctaactaagtcgttaaatttcaatcctctgaaacaacgtaccaaaagttatgaccttctgataatatttactttgcgtgcggttttgtgaaacctagcttagtcccaatattttttaataaatcagaaaacaaaattgaaagattgcatgcatggtctgcagtagctcgagaatgtggtccacacgaagttgcataatctcatatccaatcaaatttgatctacatatacgcagatatcatggttaggtagaaaatttattttggagttatttttgtgagactgtggacacctctctgcgcatctattgatttcagcattgcttctgaccagaagaagatcccggaagaaacaagatcagaaccagaaaccacagctaccgcgaagtggaagtcagatcatacatagagagcattttgaagctgtagctgttggatagaccgagctgctcgatcaatcgtgatacacccaagcgatcggccgaactgtgtagctcgcataggtagaga

SEQ ID NO: 16 - DNA, 1264 bp, Lolium perenne
ttctctacctatgcgagctacacagttcggccgatcgcttgggtgtatcacgattgatcgagcagctcggtctatccaacagctacagcttcaaaatgctctctatgtatgatctgacttccacttcgcggtagctgtggtttctggttctgatcttgtttcttccgggatcttcttctggtcagaagcaatgctgaaatcaatagatgcgcagagagggcccttatataattgggcagggccagctacggccgaggcggtggtcgtggcagtggcggattcgaaggtctcagtgcgtagtttctttcctttgttttttctccaacacacacagcaagggatggaggagacatgcgcaaaagcggccactgttttgcaaaacgctagtattagcctgtctcgtgcaccgtgcccgacatccttaagcccgaaattaacaatgaggccttggtcgcccgtgcatgcagatctaagcgccatcgtgtcgcgtagcatcatgtcttagaatgcatgagatcgatcagcccctgctcgatcgattttggggttcattattccatagagatctctggctccttgcataggtcgcgactggtcgaagagaatcgtggtgaggaccaggctcatcatgcatggacaatctcgttttggagttcgctcgtctgctctaggataacccaatcgaagtgccggcctgtgcatacaataaagcataggatatagttcttcatagctgatgaacaagttaaatagcaacaagacagatttgcatttttctcatggcaaagtaagtcacctgccactatggcggataccaatttcccaaatttacttcatctattttactccagtattttacgatgcacttggacgttacttaattatttggtagctgtgcgcgcgcgtggagatttgctatatgtggttgtagtcaaacctgtcaggtgtacgtgcttggcaaattcaaggtgtgagtgtgatcggccatgaaattcaaatgagatgtcagggcagtaacatgctacatatcccggtttctctgttttagacaaacagcaacacaccgcgatgaccgatccatctatagtctcgatcgtcagtactaccggccgtccaattttcagtgcctcccacgtgtccacagtctcacaaaaataactccaaaataaattttctacctaaccatgatatctgcgtatatgtagatcaaatttgattggatatgagattatgcaacttcgtgtggaccacattctcgagctac